SEQUENCE LISTING (16 sequences)

SEQ ID NO: 1 - DNA, 13 bp, Artificial sequence
  synthetic construct
tacccgcgtccgc

SEQ ID NO: 2 - DNA, 42 bp, Artificial sequence
  synthetic construct
tgtagcgtgaagacgacagaaagggcgtggtgcggacgcggg

SEQ ID NO: 3 - DNA, 7 bp, Artificial sequence
  synthetic construct
ccctaac

SEQ ID NO: 4 - DNA, 7 bp, Artificial sequence
  synthetic construct
taaccct

SEQ ID NO: 5 - DNA, 7 bp, Artificial sequence
  synthetic construct
cctaacc

SEQ ID NO: 6 - DNA, 7 bp, Artificial sequence
  synthetic construct
ctaaccc

SEQ ID NO: 7 - DNA, 7 bp, Artificial sequence
  synthetic construct
aacccta

SEQ ID NO: 8 - DNA, 7 bp, Artificial sequence
  synthetic construct
accctaa

SEQ ID NO: 9 - DNA, 27 bp, Artificial sequence
  synthetic construct
tgctccgtgcatctggcatcccctaac

SEQ ID NO: 10 - DNA, 27 bp, Artificial sequence
  synthetic construct
tgctccgtgcatctggcatctaaccct

SEQ ID NO: 11 - DNA, 27 bp, Artificial sequence
  synthetic construct
tgctccgtgcatctggcatccctaacc

SEQ ID NO: 12 - DNA, 27 bp, Artificial sequence
  synthetic construct
tgctccgtgcatctggcatcctaaccc

SEQ ID NO: 13 - DNA, 27 bp, Artificial sequence
  synthetic construct
tgctccgtgcatctggcatcaacccta

SEQ ID NO: 14 - DNA, 27 bp, Artificial sequence
  synthetic construct
tgctccgtgcatctggcatcaccctaa

SEQ ID NO: 15 - DNA, 20 bp, Artificial sequence
  synthetic construct
tgctccgtgcatctggcatc

SEQ ID NO: 16 - DNA, 21 bp, Artificial sequence
  synthetic construct
tgtagcgtgaagacgacagaa

(19) United States
(12) Patent Application Publication
(10) Pub. No.: US 2011/0244462 A1
(43) Pub. Date: Oct. 6, 2011

(54) Method for Estimating Telomere Length

(75) Inventors: Laila Bendix, Odense S. (DK); Steen Kølvraa, Skodstrup (DK)

(73) Assignee: Tina Holding APS, Vejle (DK)

(21) Appl. No.: 12/672,853

(22) PCT Filed: Aug. 8, 2008
(86) PCT No.: PCT/DK08/50194

§ 371 Date: May 13, 2011

(30) Foreign Application Priority Data
Aug. 10, 2007 (DK) PA 2007 01140

(51) Int. Cl. C12Q 1/68

(52) U.S. Cl. 435/6.12

(57) ABSTRACT

Knowledge about telomere length is highly relevant in cancer and age related research. Currently applied methods for determining telomere length are subject to several drawbacks preventing fast and reliable information concerning telomere length. The present invention relates to a method for determining telomere length which is fast and reliable. The method is PCR based and may advantageously be performed in a “one tube system”, whereby time consuming and inconvenient handling steps are avoided. The method comprises annealing of up- and downstream tags to telomere fragments and subsequent PCR amplification of telomere fragments using primers having a sequence complementary or identical to at least part of the up- and downstream oligonucleotide tags.

All patent and non-patent references cited in the application, or in the present application, are also hereby incorporated by reference in their entirety.

### FIELD OF INVENTION

The present invention relates to the field of telomere research wherein fast and reliable methods for determining or estimating the length of telomeres are of great interest.

### BACKGROUND OF INVENTION

Due to the “end replication problem” associated with DNA replication in eukaryotes, lagging strand shortens by DNA replication (Olovnikov, 1973). This occurs as a consequence of the mechanism of replication. During replication of the lagging strand short sequences of RNA acting as primers attach to the lagging strand a little way ahead of where the initiation site was. The DNA polymerase can start replication at that point and go to the end of the initiation site. This causes the formation of Okazaki fragments. More RNA primers attach further on the DNA strand and DNA polymerase and DNA ligase come along to convert the RNA (of the primers) to DNA and to seal the gaps in between the Okazaki fragments. But in order to change RNA to DNA, there must be another DNA strand in front of the RNA primer. This happens at all the sites of the lagging strand, except at the chromosome ends where the last RNA primer is attached. Ultimately, that RNA is destroyed by enzymes that degrade RNA left on the DNA. Thus, a section of the chromosomal DNA is lost during each cycle of replication.

### Telomere

Telomeres are specialized protein-DNA constructs present at the ends of eukaryotic chromosomes, which prevent them from degradation due to the incapability of the polymerase complex of replicating all the way to the end of the chromosome—the “end replication problem”. The telomeres further prevent end-to-end chromosomal fusion (Harley, 1990). A telomere is a region of highly repetitive DNA at the end of a linear chromosome which in humans consist long (TTAGGG)n repeats of variable length, often around 3-20 kb. There are additional 100-300 kilobases of telomere-associated repeats between the telomere and the rest of the chromosome. The region comprising these telomere associated repeats or incomplete repeats is termed the subtelomeric region. Telomere sequences vary from species to species, but are generally GC-rich.

These GC-rich sequences can form four-stranded structures (G-quadruplexes), with sets of four bases held in plane and then stacked on top of each other with either a sodium or potassium ion between the planar quadruplexes.

The telomere employ a different mechanism for DNA synthesize than the method of DNA synthesis employed during replication whereby the sequence at the terminal of the chromosome is preserved. This prevents chromosomal fraying and prevents the ends of the chromosome from being processed as a double strand DNA break, which could lead to chromosome-to-chromosome telomere fusions.

Telomeres are extended by a telomerase, which is part of a protein subgroup of specialized reverse transcriptase enzymes known as TERT (TElomerase Reverse Transcriptases) that are involved in synthesis of telomeres in humans and many other, but not all, organisms. However, because of DNA replication mechanisms and because TERT expression is repressed in many types of human cells, the telomeres in cell not expressing TERT shrink a little bit every time a cell divides although in other cellular compartments which require extensive cell division, such as stem cells and certain white blood cells, TERT is expressed and telomere length is maintained.

In addition to its TERT protein component, telomerase also contains a piece of template RNA known as the TERC (TElomerase RNA Component) or TR (Telomerase RNA) that serves as a template for the TERT mediated elongation of the telomeres (Collins, 2002)

At the very distal end of the telomere is a 300 bp single-stranded portion which forms the T-Loop. This loop is analogous to a ‘knot’ which stabilizes the telomere; preventing the telomere ends from being recognized as break points by the DNA repair machinery. Should non-homologous end joining occur at the telomeric ends, chromosomal fusion will result. The T-loop is held together by seven known proteins; most notably TRF1, TRF2, POT1, TIN1, and TIN2 (Griffith, 1999 and Blackburn, 2000).

There are theories that the steady shortening of telomeres with each replication in somatic (body) cells may have a role in senescence and in the prevention of cancer (Campisi, 1997, Allsopp, 1992, Ben-Porath, 2004, Engelhardt, 1997, Gisselsson, 2001, and Zou, 2004). This is because the telomeres act as a sort of time-delay “fuse”, eventually running out after a certain number of cell divisions.

Besides the end replication problem, there is evidence that stress, especially oxidative stress, plays a role in telomere shortening. It is supposed that stress accelerates telomere shortening because of a telomere-specific single strand break repair deficiency (Martin-Ruiz, 2004).

Loss of telomeric DNA, through repeated cycles of cell division or due to oxidative stress, is associated with senescence or somatic cell aging. In contrast, germ line and cancer cells which are immortal possess a telomerase enzyme which prevents this telomere degradation and maintains telomere integrity and it is thus believed that telomeres have a function in cancer and the ageing process.

A study published in the May 3, 2005 issue of the American Heart Association journal Circulation (Gardner, 2005) found that weight gain and increased insulin resistance were correlated with greater telomere shortening over time.

If telomeres become too short, they will potentially unfold from their presumed closed structure. It is thought that the cell detects this uncapping as DNA damage and will enter cellular senescence, growth arrest or apoptosis depending on the cell's genetic background (p53 status). Uncapped telomeres also result in chromosomal fusions. Since this damage cannot be repaired in normal somatic cells, the cell may even go into apoptosis. Many aging-related diseases are linked to shortened telomeres (Benetos, 2004, Cawthon, 2003 and Meeker, 2004). Organs deteriorate as more and more of their cells die off or enter cellular senescence.

Due to the role of telomeres in cancer and age related diseases information regarding the length of the telomeres is desired.

The most widely used methods for determine telomere length is the Telomere Restriction Fragment length assay (TRF). In this assay restriction enzyme digested chromosomal DNA is separated by gel electrophoresis followed by Southern blotting and hybridization of a probe containing the telomeric repeat sequence. Although this is one of the most used methods it suffers many drawbacks. The method requires a large amount of purified DNA, which is a limiting factor when studying many kinds of tissue samples. The most used enzymes for this method, HinfI and RsaI, do not cut the subtelomeric region and because of this a subtelomeric fraction of unknown length is included in the measure. As a consequence it has recently become apparent that the use of different restriction enzymes can lead to different length measures. The TRF-assay is biased against the shorter telomeres since these bind very few copies of the probes and thereby are not visualized well by the detection system.

Further methods for determining telomere length are based on primer extension analysis, wherein a primer is annealed to genomic DNA fragments either at the 3′ end overhang of the G-rich strand of the telomere or to the C-rich strand by use of a unique sequence identified in the chromosomal DNA out side the telomeric region, such as in the subtelomeric region. Using this approach the primer extension products may be directly labeled circumventing the need for a hybridization step.

Recently, a quantitative-PCR based method developed by Cawthon (Cawthon, 2002) has become very popular. This method only needs a small amount of DNA and is less laborious making it suitable for larger series of samples. The fact that this method only gives an estimate of the total amount of telomere repeats and not the length seems to be the main limitation of this method. It is also apparent that the outcome is very sensitive to the quality of the DNA (Koppelstaetter, 2005).

A further PCR based method for determining telomere length has been described in U.S. Pat. No. 5,834,193 wherein a single or double stranded linker is ligated to the 3′ end of the G-rich telomere strand by “blunt end” ligation, this linker may together with a unique region 5′ to the telomere serve as primer binding sites for PCR amplification of the telomere region.

A further method to measure the length of individual telomeres is named STELA and is as the above mentioned method a ligation-PCR based method (Baird, 2003) and WO 03 00927. The key feature of the STELA assay is the first step. In this step a linker is annealed to the G-rich 3′-overhang of the telomere. Afterwards this linker, called “telorette” is ligated to the 5′-end of the complementary C-rich strand. In this way the end of the telomere is tagged with a unique sequence. PCR can then be performed using a downstream primer, called ‘teltail’, complementary to the telorette tail and a chromosome specific upstream primer.

The methods described above all have several limitations. The methods dependent on direct length measurements of telomeric DNA require a large amount of DNA, which is also true for the primer extension based methods. The PCR amplification methods described above are either very imprecise or require knowledge of unique sequences useful as primers binding sites out side the telomeric region and such upstream primer binding sites have only been designed to few chromosomes. Even if the subtelomeric region of all chromosomes should be sequenced it would most likely be difficult to design telomere near and chromosome specific primers for all chromosomes, due to the fact that the human subtelomeric region contains many repeated sequences, which are highly variable and which further comprise regions shared among different chromosomes.

### SUMMARY OF INVENTION

The invention described herein relates to a method for estimating telomere length which overcomes several of these limitations associated with previously known methods.

In an aspect of the invention two ligation based steps are exploited whereby sequences suitable as primer binding sites are made available both upstream and downstream (see FIG. 1) of the telomeric DNA region. This allows amplification of the telomeric DNA fragments, followed by determination of the length of the amplified product.

In an embodiment the invention relates to a method for estimating telomere length comprising the following steps:
a. digestion of a genomic DNA preparation generating telomere fragments
b. ligation of an up-stream oligonucleotide tag to the telomere fragments
c. ligation of a down-stream oligonucleotide tag to the telomere fragments,
d. amplification of telomere fragments using primers with a sequence complementary or identical to at least part of the up- and downstream oligonucleotide tags obtaining amplified telomere fragments and
e. estimate telomere length by determining the length of the amplified telomere fragments.

Initially the chromosomal DNA is digested with restriction enzyme(s), preferably cutting the chromosomal DNA in the subtelomeric regions. It is further preferred that the digest is performed with enzyme(s) which are frequent cutters, so that the chromosomal DNA is cut into fragments of less than 3000 bp.

The restriction enzyme(s) is/are preferably selected to leave an overhang such as a two-base sticky overhang upstream of the telomere repeat. This may in the following step guide annealing of the upstream oligonucleotide tag having a complementary overhang. In this preferred embodiment the upstream oligonucleotide tag has an overhang, due to one end of the double-oligo matching the ends of the digested DNA. The other end of the double oligo is designed so that the DNA is tagged with a non-complementary sequence.

A downstream oligonucleotide tag is covalently bound to the down stream end of the telomeric fragments and as the upstream oligonucleotide tag, this oligo also includes a non-complementary sequence. Using the non-complementary sequence, not being identical, attached in each end of the telomere fragments as binding sites for PCR primers the telomere fragments can be amplified. Depending on the specific sequences of the oligonucleotide tags, the primers should be either identical or complementary in sequence to at least a part of the non-complementary sequence.

In order to have a higher specificity the down-stream oligonucleotide tag and the primer used for PCR may have the sequences as described in (Baird, 2003), wherein the STELA assay mentioned above is described.

In a preferred embodiment the amplification is performed by the polymerase chain reaction (PCR).

In a preferred embodiment the method according to the invention comprise the following steps:
a) digestion of a genomic DNA preparation generating telomere fragments
b) annealing of an up-stream oligonucleotide tag to the telomere fragments and ligation of the up-stream oligonucleotide tag to the telomere fragments
c) annealing of a down-stream oligonucleotide tag to the telomer fragments and ligation of the down-stream oligonucleotide tag to the telomere fragments,
d) PCR amplification of telomere fragments using primers with a sequence complementary or identical to at least part of the up- and downstream oligonucleotide tags obtaining amplified telomere fragments and
e) estimate telomere length by determining the length of the amplified telomere fragments.

In order to preferentially amplify the telomere fragments the down stream oligonucleotide tag may be designed to stimulate the formation of a panhandle loop when present on both ends of DNA fragments. This technique is known as suppression PCR (Lavrentieva, 1999), and will thus suppress PCR products from fragments that have the upstream olignucleotide tag attached in both ends.

This method is independent on the sequence of the individual chromosomes and thus overcome the problem of designing the specific upstream primers.

By minimizing handling and loss of DNA the method can thus be applied to large series of samples and using very small amounts of material.

Due to the amplification step only small amounts of DNA is required. The method may be performed using 5 pg-1 ng digested and ligated DNA

The inventors have identified conditions that allow step a) to c) to be performed in a one-tube system and with no intermediate precipitation or purification steps.

The method according to the invention gives an estimate of the mean telomeric length as well as the distribution of the short telomeres from all chromosomes.

### DESCRIPTION OF DRAWINGS

FIG. 1. Overview of method. A detailed overview of the assay principles is described in the examples.

FIG. 2. Validation of method. The amplification step is performed using up- and down stream primers identified by SEQ ID NO 15 and 16 using templates having covalently bound different combinations of up- and down-stream oligonucleotide tags. No PCR product is produced when no tags (dig)—lane 1-2, only a down stream tag (telorette (tel))—lane 3-4 or only a upstream oligonucleotide tag (panhandle (pan))—lane 7-8 is/are ligated to the digested DNA. A PCR product is only achieved when using a template (telomere fragments) that has been ligated to both the up- and down-stream oligonucleotide tags (panhandle and telorette). A PCR product is achieved using both a separate (fill-in)—lane 9-10 as well as a build-in fill-in step (t+p)—lane 5-6.

FIG. 3. Evaluation of downstream oligonucleotide tag specificity. The second ligation step has been done with the down stream oligonucleotide tags 1-6 (SEQ ID NO 9-14) in separate tubes. It has earlier been shown (Sfeir, 2005) that app. 80% of the telomeres can be detected using downstream oligonucleotide tag 11. The same biological distribution is shown using the method according to the invention. Six reactions are run per downstream oligonucleotide tag. The DNA preparation is obtained from a whole blood sample.

FIG. 4. Sensitivity to template amount. Southern blot of amplified telomere fragments according to the invention using different amounts of template (a). Graphical view of the mean length of telomere amplification products versus the amount of template used (b). The method according to the invention is very sensitive to the amount of template used. When using high amounts (>1 ng) of template DNA the amplification is almost fully suppressed. When using intermediate amounts of template (0.3-1 ng) a smear is seen probably representing a network of unfinished PCR products. Distinct bands are seen with 5 pg-200 pg. The sharpest bands are seen with 20-40 pg. Using 75-200 pg a smaller estimate of the mean length is obtained. Using DNA from single cells (5-10 pg) the variation is relatively high.

FIG. 5. The relationship between telomere lengths determined by TRF assay and estimated by the method of the present invention (here called UniSTELA). The relationship between the TRF length and the mean length estimated using the method according to the invention should in theory be 1 (α=1). For the telomere fragments determined by TRF being less than approximately 6.5 kb, the relationship comes close to being linear, with a slope of 0.63 (α=0.63) and not 1. This discrepancy is due to the fact that the TRF assay is insensitive to picking up the shorter telomere fragments, thereby overestimating the length, while the method according to the invention favors the shorter telomere fragments thereby underestimating the length. This is clear when analyzing samples with very long telomere fragments were the curve becomes horizontal.

FIG. 6. Telomere distributions in ALT negative and positive cells. This figure shows how the method according to the invention can describe the biological difference between a normal fibroblast cell line (WI38) and an immortalized ALT positive subpopulation of the same cell line (WI38 ALT). For the fibroblast cell line there is only few very short telomere, while there is a long tail of short telomeres in the WI38ALT cell line.

FIG. 7. Distribution of telomeres in telomerase positive cells with different mean lengths. The means of the telomere length estimated as described in the example is depicted by an X, and the TRF length is depicted by an O. In all samples short telomeres are found.

### DETAILED DESCRIPTION OF THE INVENTION

### Definitions

Amplification: amplification according to the present invention is the process wherein a plurality of exact copies of a starting molecule is synthesised, without employing knowledge of the exact composition of the starting molecule. Hence a template may be amplified even though the exact composition of said template is unknown. In one preferred embodiment of the present invention amplification of a template comprises the process wherein a template is copied by a nucleic acid polymerase or polymerase homologue, for example a DNA polymerase or an RNA polymerase. For example, templates may be amplified using reverse transcription, the polymerase chain reaction (PCR), ligase chain reaction (LCR), in vivo amplification of cloned DNA, and similar procedures capable of complementing a nucleic acid sequence.

Annealing: Annealing and hybridization are used interchangeable. Annealing covers the process of binding together two oligo- or poly-nucleotides by the force of the hydrogen bonds between the complementary nucleotide bases. Annealing is mostly used for the binding of a primer to a target nucleotide sequence.

Anticodon: a sequence of 3 ribonucleotides that can pair with the bases of a corresponding codon on a messenger RNA.

Base: Nitrogeneous base moiety of a natural or non-natural nucleotide, or a derivative of such a nucleotide comprising alternative sugar or phosphate moieties. Base moieties include any moiety that is different from a naturally occurring moiety and capable of complementing one or more bases of the opposite nucleotide strand of a double helix. In this context a base refers to one of the bases in nucleic acid or modified nucleic acid unless otherwise noted. The bases of DNA, for example are adenosine, cytidine, guanosine, and thymidine.

Chimeric polynucleotide: Polynucleotide comprising an oligonucleotide part that is ligated to a polynucleotide derived from a biological sample. A chimeric polynucleotide can also be a single stranded polynucleotide. The polynucleotide derived from a biological sample can also be a truncated part of a polynucleotide obtained from a biological sample. Chimeric polynucleotide also denotes any cDNA copy of a chimeric RNA polynucleotide.

Cleavage agent: Agent capable of recognizing a predetermined motif of a double stranded polynucleotide and cleaving only one strand of the double stranded polynucleotide, or capable of cleaving both strands of the double stranded polynucleotide. Examples of cleavage agents in the present context is type II restriction endonucleases, type IIs restriction endonucleases, and nicking endonucleases having activities as outlined e.g. in New England BioLabs' catalog for 2000-01. The term digestion also relates to the cleavage of single or doublestranded polynucleotide molecules, such as DNA molecules.

Codon: A codon is a sequence of 3 ribonucleotides that encodes a particular amino acid in a messenger RNA molecule.

DNA: deoxyribonucleic acid.

Complementary and substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Complementary nucleic acid sequences hybridize over the entire length of the complementary region. Oligonucleotide primers may comprise a non-complementary region designed for various specific purposes.

Two single stranded RNA or DNA molecules are said to be substantially complementary (in a defined region) when the nucleotides of one strand, optimally aligned and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Selective hybridization conditions include, but is not limited to, stringent hybridization conditions. Selective hybridization may occur when there is at least about 65% complementarity over a stretch of at least 14 to 25 nucleotides, preferably at least about 75%, more preferably at least about 90% complementarity. For shorter nucleotide sequences selective hybridization occurs when there is at least about 65% complementarity over a stretch of at least 8 to 12 nucleotides, preferably at least about 75%, more preferably at least about 90% complementarity. Stringent hybridization conditions will typically include salt concentrations of less than about 1 M, more usually less than about 500 mM and preferably less than about 200 mM. Hybridization temperatures can be as low as 5° C. and are preferably lower than about 30° C. However, longer fragments may require higher hybridization temperatures for specific hybridization. Hybridization temperatures are generally about 2° C. to 6° C. lower than melting temperatures. As other factors may affect the stringency of hybridization, including base composition and length of the complementary strands, presence of organic solvents and extent of base mismatching, the combination of parameters is more important than the absolute measure of any one alone.

Complementary strand: Double stranded polynucleotide contains two strands that are complementary in sequence and capable of hybridizing to one another.

Complementary DNA (cDNA): Any DNA obtained by means of reverse transcriptase acting on RNA as a substrate. Complementary DNA is also termed copy DNA.

Digest is used interchangeable with cleavage.

Double stranded polynucleotide: Polynucleotide comprising complementary strands.

Double stranded oligo-nucleotide tag: oligo-nucleotide tags as described below may be single of double stranded. Double stranded oligonucleotide tags comprise complementary strands of consecutive nucleotides linked together in two individual strands. The number of nucleotides in each strand may range from about 10, such as 15, for example 20, such as 25, for example 30 nucleotides, to more than 50 nucleotides, including oligonucleotide tags of more than e.g. 200 nucleotides. The length of the two individual strands of the double-stranded oligonucleotide tag may be different, giving rise to an overhang in one or more ends of the double-stranded oligonucleotide tag. The tag sequence may be present in any one or both of the strands of the double stranded oligo-nucleotide tag.

dsDNA: Double stranded DNA.

Filling in: Single stranded regions of a DNA molecule may be rendered double stranded by filling in the gaps or open ends using a polymerase. A suitable 3′end is required due to the directional specificity of the polymerase.

Ligase (DNA-ligase): An enzyme capable of joining two polynucleotides by forming a new chemical bond. A DNA-ligase can thus link an annealed primer to a neighbouring polynucleotide sequence.

Ligation: The reaction (catalysed by a ligase) of joining two or more polynucleotides.

Melting temperature (Tm): The melting temperature is the temperature where an oligonucleotide dissociates from the target nucleic acid sequence. For primers Tm can be calculated as Tm=4×(G+C content)+2×(A+T content) including only bases pairing with nucleotides of the primer binding site. Tm may also be used in connection with double stranded regions to estimate the strength of the duplex.

Messenger RNA (mRNA): mRNA, a polynucleotide being transcribed only from genes that are actively expressed, where the expressed mRNA codes for a protein.

Natural nucleotide: Any of the four deoxyribonucleotides, dA, dG, dT, and dC (constituents of DNA), and the four ribonucleotides, A, G, U, and C (constituents of RNA) are the natural nucleotides. Each natural nucleotide comprises or essentially consists of a sugar moiety (ribose or deoxyribose), a phosphate moiety, and a natural/standard base moiety. Natural nucleotides bind to complementary nucleotides according to well-known rules of base pairing where adenine (A) pairs with thymine (T) or uracil (U); and where guanine (G) pairs with cytosine (C), wherein corresponding base-pairs are part of complementary, anti-parallel nucleotide strands. The base pairing results in a specific hybridization between predetermined and complementary nucleotides. The base pairing is the basis by which enzymes are able to catalyze the synthesis of an oligonucleotide complementary to the template oligonucleotide. In this synthesis, building blocks (normally the triphosphates of ribo or deoxyribo derivatives of A, T, U, C, or G) are directed by a template oligonucleotide to form a complementary oligonucleotide with the correct, complementary sequence. The recognition of an oligonucleotide sequence by its complementary sequence is mediated by corresponding and interacting bases forming base pairs. In nature, the specific interactions leading to base pairing are governed by the size of the bases and the pattern of hydrogen bond donors and acceptors of the bases. A large purine base (A or G) pairs with a small pyrimidine base (T, U or C). Additionally, base pair recognition between bases is influenced by hydrogen bonds formed between the bases. In the geometry of the Watson-Crick base pair, a six membered ring (a pyrimidine in natural oligonucleotides) is juxtaposed to a ring system composed of a fused, six membered ring and a five membered ring (a purine in natural oligonucleotides), with a middle hydrogen bond linking two ring atoms, and hydrogen bonds on either side joining functional groups appended to each of the rings, with donor groups paired with acceptor groups.

Non-natural base pairing: Base pairing among non-natural nucleotides, or among a natural nucleotide and a non-natural nucleotide. Examples are described in U.S. Pat. No. 6,037,120, wherein eight non-standard nucleotides are described, and wherein the natural base has been replaced by a non-natural base. As is the case for natural nucleotides, the non-natural base pairs involve a monocyclic, six membered ring pairing with a fused, bicyclic heterocyclic ring system composed of a five member ring fused with a six membered ring. However, the patterns of hydrogen bonds through which the base pairing is established are different from those found in the natural AT, AU and GC base pairs. In this expanded set of base pairs obeying the Watson-Crick hydrogen-bonding rules, A pairs with T (or U), G pairs with C, iso-C pairs with iso-G, and K pairs with X, H pairs with J, and M pairs with N. Nucleobases capable of base pairing without obeying Watson-Crick hydrogen-bonding rules have also been described (Berger et al., 2000, Nucleic Acids Research, 28, pp. 2911-2914).

Non-natural nucleotide: Any nucleotide not falling within the definition of a natural nucleotide.

“Nucleic acid probes” are prepared based on the cDNA sequences which encode the target sequence. Nucleic acid probes comprise portions of the sequence having fewer nucleotides than about 6 kb, usually fewer than about 1 kb. After appropriate testing to eliminate false positives, these probes may be used to determine whether the target sequence such as the target mRNA is present in a cell or tissue or to isolate similar nucleic acid sequences from chromosomal DNA extracted from such cells or tissues as described by Walsh et al. (Walsh, 1992). Probes may be derived from naturally occurring or recombinant single- or double-stranded nucleic acids or be chemically synthesized. They may be labeled by nick translation, Klenow fill-in reaction, PCR or other methods well known in the art such as in Sambrook et al., 1989 or Ausubel et al., 1989.

Nucleoside: A base attached to a ribose ring, as in RNA nucleosides, or a deoxyribose ring, as in DNA nucleosides. See also: “Base”.

Nucleotide: Monomer of RNA or DNA components. A nucleotide is a ribose or a deoxyribose ring attached to both a base and a phosphate group. Both mono-, di-, and tri-phosphate nucleosides are referred to as nucleotides.

Nucleotide: Nucleotides as used herein refers to both natural nucleotides and non-natural nucleotides capable of being incorporated—in a template-directed manner—into an oligonucleotide, preferably by means of an enzyme comprising DNA or RNA dependent DNA or RNA polymerase activity, including variants and functional equivalents of natural or recombinant DNA or RNA polymerases. Corresponding binding partners in the form of coding elements and complementing elements comprising a nucleotide part are capable of interacting with each other by means of hydrogen bonds. The interaction is generally termed “base-pairing”. Nucleotides may differ from natural nucleotides by having a different phosphate moiety, sugar moiety and/or base moiety. Nucleotides may accordingly be bound to their respective neighbour(s) in a template or a complementing template by a natural bond in the form of a phosphodiester bond, or in the form of a non-natural bond, such as e.g. a peptide bond as in the case of PNA (peptide nucleic acids).

Nucleotides: nucleotides according to the invention includes ribonucleotides comprising a nucleobase selected from the group consisting of adenine (A), uracil (U), guanine (G), and cytosine (C), and deoxyribonucleotide comprising a nucleobase selected from the group consisting of adenine (A), thymine (T), guanine (G), and cytosine (C). Nucleobases are capable of associating specifically with one or more other nucleobases via hydrogen bonds. Thus it is an important feature of a nucleobase that it can only form stable hydrogen bonds with one or a few other nucleobases, but that it can not form stable hydrogen bonds with most other nucleobases usually including itself. The specific interaction of one nucleobase with another nucleobase is generally termed “base-pairing”. The base pairing results in a specific hybridisation between predetermined and complementary nucleotides. Complementary nucleotides according to the present invention are nucleotides that comprise nucleobases that are capable of base-pairing. Of the naturally occurring nucleobases adenine (A) pairs with thymine (T) or uracil (U); and guanine (G) pairs with cytosine (C). Accordingly, e.g. a nucleotide comprising A is complementary to a nucleotide comprising either T or U, and a nucleotide comprising G is complementary to a nucleotide comprising C.

Nucleotide analog: Nucleotide capable of base-pairing with another nucleotide, but incapable of being incorporated enzymatically into a template or a complementary template. Nucleotide analogs often includes monomers or oligomers containing non-natural bases or non-natural backbone structures that do not facilitate incorporation into an oligonucleotide in a template-directed manner. However, interaction with other monomers and/or oligomers through specific base pairing is possible. Alternative oligomers capable of specifically base pairing, but unable to serve as a substrate of enzymes, such as DNA polymerases and RNA polymerases, or mutants or functional equivalents thereof, are defined as nucleotide analogs herein. Oligonucleotide analogs includes e.g. nucleotides in which the phosphodiester-sugar backbone of natural oligonucleotides has been replaced with an alternative backbone include peptide nucleic acid (PNA), locked nucleic acid (LNA), and morpholinos.

Nucleotide derivative: Nucleotide or nucleotide analog further comprising an appended molecular entity. Often, derivatized building blocks (nucleotides to which a molecular entity have been appended) can be enzymatically incorporated into oligonucleotides by RNA or DNA polymerases, using as substrate the triphosphate of the derivatized nucleoside. In many cases such derivatized nucleotides are incorporated into the growing oligonucleotide chain with high specificity, meaning that the derivative is inserted opposite a predetermined nucleotide in the template. Such an incorporation will be understood to be a specific incorporation. The nucleotides can be derivatized on the bases, the ribose/deoxyribose unit, or on the phosphate. Preferred sites of derivatization on the bases include the 8-position of adenine, the 5-position of uracil, the 5- or 6-position of cytosine, and the 7-position of guanine. The nucleotide-analogs described below may be derivatized at the corresponding positions (Benner, U.S. Pat. No. 6,037,120). Other sites of derivatization may be used, as long as the derivatization does not disrupt base pairing specificity. Preferred sites of derivatization on the ribose or deoxyribose moieties are the 5′, 4′ or 2′ positions. In certain cases it may be desirable to stabilize the nucleic acids towards degradation, and it may be advantageous to use 2′-modified nucleotides (U.S. Pat. No. 5,958,691). Again, other sites may be employed, as long as the base pairing specificity is not disrupted. Finally, the phosphates may be derivatized. Preferred derivatizations are phosphorothiote. Nucleotide analogs (as described below) may be derivatized similarly to nucleotides. It is clear that the various types of modifications mentioned herein above, including i) derivatization and ii) substitution of the natural bases or natural backbone structures with non-natural bases and alternative, non-natural backbone structures, respectively, can be applied once or more than once within the same molecule.

Oligonucleotide: Used herein interchangeably with polynucleotide. The term oligonucleotide comprises oligonucleotides of both natural and/or non-natural nucleotides, including any combination thereof. The natural and/or non-natural nucleotides may be linked by natural phosphodiester bonds or by non-natural bonds.

Oligonucleotide: The oligomer or polymer sequences of the present invention are formed from the chemical or enzymatic addition of monomer nucleotide subunits. When nucleotides are conjugated together in a string using synthetic procedures, they are always referred to as oligo-nucleotides (or oligo for short). The term “oligonucleotide” as used herein includes linear oligomers of natural or modified monomers, including deoxyribonucleotides, ribonucleotides, anomeric forms thereof, peptide nucleic acid monomers (PNAs), locked nucleotide acid monomers (LNA), and the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′ to 3′ order from left to right and the “A” denotes deoxyadenosine, “C” denotes deoxycytidine, “G” denotes deoxyguanosine, and “T” denotes thymidine, unless otherwise noted. Usually oligonucleotides of the invention comprise the four natural nucleotides; however, they may also comprise methylated or non-natural nucleotide analogs. Suitable oligonucleotides may be prepared by the phosphoramidite method described by Beaucage and Carruthers (Tetrahedron Lett., 22, 1859-1862, 1981), or by the triester method according to Matteucci, et al. (J. Am. Chem. Soc., 103, 3185, 1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical configuration typically associated with, for example, DNA. In addition to the 100% complementary form of double-stranded oligonucleotides, the term “double-stranded” as used herein is also meant to refer to those forms which include such structural features as bulges and loops. For example as described in U.S. Pat. No. 5,770,722 for a unimolecular double-stranded DNA. It is clear to those skilled in the art when oligonucleotides having natural or non-natural nucleotides may be employed, e.g. where processing by enzymes is called for, usually oligonucleotides consisting of natural nucleotides are required.

Oligonucleotide tag: For the present application an oligonucleotide tag is a single or doublestranded oligonucleotide which comprises a sequence tag. A tag is a handle for subsequent analysis of nucleotide sequences to which the oligonucleotide tag has been bound. The sequence of the tag may provide one or more special features to the oligonucleotide, examples are restriction endonuclease recognition sites and primer annealing sites.

Polynucleotide: A plurality of individual nucleotides linked together in a single molecule. Polynucleotide covers any derivatized nucleotides such as DNA, RNA, PNA, LNA etc. Any oligonucleotide is also a polynucleotide, but every polynucleotide is not an oligonucleotide.

Primer: An oligonucleotide or polynucleotide designed to hybridize (bind) to a target nucleic acid sequence through hydrogen bonds. The primer may subsequently be extended by the addition of nucleotides or oligonucleotides. This addition is often performed by a polymerase or a ligase.

Ribose derivative: Ribose moiety forming part of a nucleoside capable of being enzymatically incorporated into a template or complementing template. Examples include e.g. derivatives distinguishing the ribose derivative from the riboses of natural ribonucleosides, including adenosine (A), guanosine (G), uridine (U) and cytidine (C). Further examples of ribose derivatives are described in e.g. U.S. Pat. No. 5,786,461. The term covers derivatives of deoxyriboses, and analogously with the above-mentioned disclosure, derivatives in this case distinguishes the deoxyribose derivative from the deoxyriboses of natural deoxyribonucleosides, including deoxyadenosine (dA), deoxyguanosine (dG), deoxythymidine (dT) and deoxycytidine (dC).

RNA: ribonucleic acid. Different groups of ribonucleic acids exists: mRNA, tRNA, rRNA and nRNA.

Sequence determination: Used interchangeably with “determining a nucleotide sequence” in reference to polynucleotides and includes determination of partial as well as full sequence information of the polynucleotide. That is, the term includes sequence comparisons, fingerprinting, and like levels of information about a target polynucleotide, as well as the express identification and ordering of bases, usually each base, in a target polynucleotide. The term also includes the determination of the identification, ordering, and locations of one, two, or three of the four types of nucleotides within a target polynucleotide. For example, in some embodiments sequence determination may be effected by identifying the ordering and locations of a single type of nucleotide, e.g. cytosines, within the target polynucleotide “CATCGC . . . ” so that its sequence is represented as a binary code, e.g. “100101 . . . ” for “C-(not C)-(not C)-C-(not C)-C . . . ” and the like.

Single stranded oligo-nucleotide tag: oligo-nucleotide tags as described above may be single or double stranded. Single stranded oligonucleotide tags are consecutive nucleotides linked together forming a single strand. The number of nucleotides may range from about 10, such as 15, for example 20, such as 25, for example 30 nucleotides, to more than 50 nucleotides, including oligo-nucleotide tags of more than 200 nucleotides.

Site-specific cleavage agent: Any agent capable of recognising a predetermined nucleotide motif and cleaving a single stranded nucleotide and/or a double stranded nucleotide. The cleavage may occur within the nucleotide motif or at a location either 5′ or 3′ to the nucleotide motif being recognised.

Site-specific endonuclease: Enzyme capable of recognizing a double stranded polynucleotide and cleaving only one strand of the double stranded polynucleotide, or capable of recognizing a double stranded polynucleotide and cleaving both strands of the double stranded polynucleotide. One group of site-specific endonucleases is blocked in their activity by the presence of methylated bases in specific position in their recognition sequence. Another group of site-specific endonucleases is dependant upon methylated bases in specific position in their recognition sequence. A third group of site-specific endonucleases are oblivious to methylated bases in specific positions in their recognition sequence.

Site-specific Restriction Endonuclease: Enzyme capable of recognizing a double stranded polynucleotide and cleaving both strands of the double stranded polynucleotide. Examples of site-specific restriction endonucleases are shown in New England BioLabs' catalog for 2007-08.

Site-specific Nicking Endonuclease: Enzyme capable of recognizing a double stranded polynucleotide and cleaving only one strand of the double stranded polynucleotide. An example of site-specific nicking endonucleases is shown in New England BioLabs' catalog for 2007-08.

ssDNA: Single stranded DNA.

ssDNA tag: Single-stranded polynucleotide tag comprising, or essentially consisting of, or consisting exclusively of a single strand of consecutive deoxyribonucleic acids.

Sticky ends: Polynucleotides having complementary 3′ and 5′ ends that are capable of holding the two polynucleotides linked together by the force of the hydrogen bonds between the complementary overhangs are said to have sticky ends.

Strand: Stretch of individual nucleotides linked together and forming an oligonucleotide or a polynucleotide. Normally a strand denotes a single stranded polynucleotide such as ssDNA or RNA. See “Double stranded polynucleotide”.

Up-stream and down-stream is herein used in connection with oligonucleotide tags and primers. The terms are used to define the position of an oligonucleotide tag/primer binding site relative to a defined region. In relation to transcription units upstream, denotes the region to the left of the +1 (or towards the 5′ end) transcription initiation site and downstream, denotes the region to the right (or towards the 3′) of the termination site. For the present invention up-stream and down-stream refers to the telomeric repeat region, thus an upstream oligonucleotide tag binds to DNA in the subtelomeric region and the down-stream oligonucleotide tag primer binds to the 3″-overhang.

Telomere fragment: DNA fragments comprising the telomeric region of the chromosomes. Telomere fragments may further comprise some subtelomeric region.

Telomeric DNA: Each end of the chromosomes consists of a region of repeated nucleotide sequences. In human telomeric regions the telomere repeat sequence is 5′-TTAGGG-3′ and the complementary sequence. The sequence of the telomere repeat sequence in different species can be seen in table 1.

Subtelomeric DNA: The region located adjacent to the telomeric repeats. This region, besides the telomere repeat also comprise repeats of the telomere variant sequences such as 5′-TGAGGG-3′, 5′-TCAGGG-3′ and 5′-TTGGGG-3′. The variant sequences are found in the telomeric region furthest from the chromosome ends and include the most distal (furthest from the centromere) region of unique DNA on a chromosome.

### DESCRIPTION OF THE INVENTION

The present invention relates to a method for estimating telomere length.

As described in the background section, telomeres comprise long stretches of repetitive DNA, which make molecular analysis of this region difficult. Due to the specific repeat sequence, digestion of chromosomal DNA using enzymes which does not target the repeat sequence will leave the telomeric DNA unchanged. The subtelomeric sequence of the chromosome ends is less well defined and may thus be cut by some restriction enzymes. Digestion of a genomic DNA preparation will result in the formation of DNA fragments of various sizes whereof two from each chromosome will comprise the telomeric DNA, these fragments are herein described as telomere fragments, although depending on the enzyme used for digestion, also subtelomeric DNA may be present in the telomere fragments.

In order to determine telomere length using small amounts of starting material, amplification of the telomere fragments is desirable.

For this purpose the telomere fragments are according to the invention tagged with both an up stream and a down stream oligonucleotide tag. These tags allow amplification of the telomere fragments, and consequently the estimation of the lengths of the telomere fragments.

An aspect of the invention relates to a method for estimating telomere length comprising the following steps:
a) digestion of a genomic DNA preparation generating telomere fragments
b) ligation of an up-stream oligonucleotide tag to the telomere fragments
c) ligation of a down-stream oligonucleotide tag to the telomere fragments,
d) amplification of telomere fragments using primers with a sequence complementary or identical to at least part of the up- and downstream oligonucleotide tags obtaining amplified telomere fragments and
e) estimate telomere length by determining the length of the amplified telomere fragments.

### Genomic DNA Preparation

The genomic DNA preparation may be obtained from any type of sample, such as a blood sample or tissue sample, wherefrom knowledge of telomere length is sought.

Such DNA preparations can be prepared by any suitable method know in the art, such as the common desalting procedure or any commercially available kit.

### Restriction Enzyme Digest

In order to create an upstream ligation site, the genomic DNA preparation is treated with a restriction enzyme. The most commonly used enzymes are type II restriction endonucleases which have activities as outlined in the New England Biolab's catalog for 2007-8. Type II restriction endonucleases cleave both strands of the DNA at very specific sites that are within or close to their recognition sequence. A plurality of restriction enzymes (type II restriction endonucleases) is available through several companies such as new New England Biolab. Based on the knowledge of the telomeric repeat sequence, it is possible to identify restriction enzymes that do not cleave the telomeric DNA. In the absence of precise sequence information related to the subtelomeric regions the length of any subtelomeric DNA can not be deduced without experimentation. The telomere fragments comprising the telomeric DNA may therefore further comprise sub-telomeric DNA of unknown length.

The method according to the invention is aimed at estimating the length of the telomeric DNA, and thus it is desirable to use restriction enzymes that cleave either in the proximal, imperfect telomeric repeats or within the subtelomeric DNA, in order to generate telomere fragments comprising a high fraction of perfect telomeric repeats.

In an embodiment of the invention one or more restriction enzymes which cut in or close to the subtelomeric region are employed.

It is further preferred that the restriction enzymes are capable of cleaving the reminder of the chromosomal DNA into small fragments, that is DNA fragments of an averages length of 100-5000 bp, or such as less than approximately 3000 bp.

The sequences of the subtelomeric regions and the chromosomal DNA are different from chromosome to chromosome and from one end to the other end of a specific chromosome. Thus in order to obtain efficient digest of different chromosome and subtelomeric sequences a combination of one or more enzymes may be used. In a preferred embodiment a mix of enzymes is used.

In an embodiment of the invention step a) of the method as outlined above is preferably performed using one or more restriction enzymes which cut close to or in the subtelomeric region. It is further preferred that the one or more restriction enzymes are frequent cutters, that is an enzyme with a recognition site present frequently in the chromosomal DNA, most likely a four base pair recognition site, which will cleave the non-telomeric and non-subtelomeric chromosomal DNA into fragments of an average length of 100-500 bases.

The TFR assay has been shown to be sensitive to the restriction enzymes used (Baird, 2006), and the applicant have investigated the effect of using three different combinations of restriction enzymes as described in the example. The HinfI/RsaI mix is known to cut outside the subtelomeric region and the HphI/MnlI mix is known for cutting in regions containing imperfect telomeric repeats located close to the perfect repeats as HphI and MnlI recognize the telomere repeat variants TGAGGG and TCAGGG respectively. In addition to the above mentioned enzymes, the applicant tested the mix of MseI and NdeI, and found that the TRF assay produced significantly shorter (˜0.8 kb) fragments using the MseI/NdeI mix than the original HinfI/RsaI mix, and only slightly longer (˜0.4 kb) fragments than when using the MnII/HphI mix.

In a preferred embodiment the digestion is performed with a mix of restriction enzymes more preferred a mix of MseI and NdeI as these enzymes, as described above, provides telomere fragments which comprise a shorter sub-telomeric DNA sequence.

The TFR assay is thus a suitable method for evaluating the usability of enzyme combination, which may be applied by the skilled person, although any other suitable technique known in the art can be applied as well.

It is clear that the features of the mix of enzymes applied may be held by one or more of the enzymes. E.g. one enzyme may cut in the subtelomeric region and one enzyme may be a frequent cutter. It may also be that more than one of the enzymes has the described features.

In a subsequent step of the method according to the invention, e.g. step b) of the overall procedure as outlined above, the upstream oligonucleotide tag (se below, section related to upstream oligonucleotide tag) is ligated to the telomere fragments. This ligation is to occur between a telomere fragment generated by the digestion as described above, and a synthetic oligonucleotide.

In specific preferred embodiments where the upstream oligonucleotide tag is double stranded the ligation reaction b) is optimised by the presence of corresponding overhangs. Thus in a preferred embodiment the digestion is performed with one or more restriction enzymes which give rise to an overhang, said overhang may be a 3′ or a 5′ overhang, and said overhang may further be a 2 or 4 bases overhang.

In a more preferred embodiment a mix of enzyme leaving a 2 base overhang is used.

The MseI and NdeI enzymes have a 4 and 6 base pair long recognition site respectively, and produce identical two base sticky overhangs e.g. a 5′ overhang of 2 bases, 5′-TA-. Thus the MseI/NdeI mix is highly preferred according to the present invention.

It is clear that further suitable combinations of enzymes may be derived by the skilled person based on the selection criteria as outlined above.

In the specific embodiment where the upstream oligonucleotide tag is single stranded (or doublestranded but designed not to form an overhang) there is no incitement to use enzymes providing overhangs, thus in a further embodiment the digestion is performed with one or more restriction enzymes which give rise to blunt ends.

### Up-Stream and Down-Stream Oligonucleotide Tags

For the present application the terms up-stream and down-stream are used in relation to a telomeric repeat region. Thus, up-streams means towards the centromer and down-streams means towards the 3′-overhang. Each chromosome comprise two telomeric repeat regions each having a single stranded telomere tail which constitutes the 3′ end of that telomere.

According to the present invention the up-stream and down-stream oligonucleotide tags are oligonucleotide constructed to enable amplification of the telomere fragment to which they are ligated. This is optimized by constructing the up-stream and down-stream oligonucleotide tags with this purpose in mind.

### Upstream Oligo-Nucleotide Tag

In the method of the invention an upstream oligonucleotide tag is ligated to the end of the telomere fragment generated by the digestion of the genomic DNA.

Besides the telomere fragments the digestion reaction also generates intra chromosomal DNA fragments with the same overhang as the upstream end of the telomere fragments. These intra-chromosomal DNA fragments will thus be equally efficient targets for ligation with the up-stream oligonucleotide tag as the telomere fragments. If no special interest is taken in the design of the up-stream oligonucleotide tag it is clear that the amplification of the telomere fragments in the later step will be hampered by the vast majority of intra-genomic fragments having the upstream oligo-nucleotide tag sequence ligated to both ends.

In order to overcome this problem, up-stream oligonucleotide tags capable of suppressing amplification of non-telomeric DNA fragments, having the upstream oligo-nucleotide tag in both ends, are preferred. In a preferred embodiment the upstream oligo-nucleotide tag suppress amplification of DNA fragments having the upstream oligo-nucleotide tag ligated to both ends.

In the example described herein the up-stream oligonucleotide tag is a set of two oligonucleotides capable of base paring with each other. The two oligonucleotides may thus anneal to each other forming a double stranded region. In a preferred embodiment the up-stream oligonucleotide tag is double stranded. A double stranded up-stream oligonucleotide need not be doublestranded over the entire length, but may comprise both single and double stranded regions.

In a different embodiment the up-stream oligonucleotide tag is single stranded.

In an embodiment the upstream oligonucleotide tag has an overhang in at least one end, said overhang being preferably complementary to the overhang created by the enzymatic digest formed by step a. If restriction enzymes leaving blunt telomeric fragments are used, the up-stream oligonucleotide tag should also be blunt in the end to be ligated to the telomeric fragment.

The sequence of oligonucleotides of the up-upstream oligonucleotide tag are determinant for there function. When annealed to each other a region of double-stranded DNA (provided that the oligos is made of dNTPs) is formed.

In a preferred embodiment of the invention the double stranded region of the upstream oligonucleotide tag is preferably at least 5 base pair long and at most 20 base pairs long, preferably the double stranded region covers 8-15 base pairs, such as 10-12 base pairs and most preferably 11 base pairs. The base content of the double stranded region influences the Tm of the region and thus it is preferred that the region has a high CG content, whereby a stable region with a relatively high Tm is formed. Preferably the CG content is at least 50% more preferably above 60% such as above 70% or 80%. If the content of CG is lower a longer double stranded region is preferred.

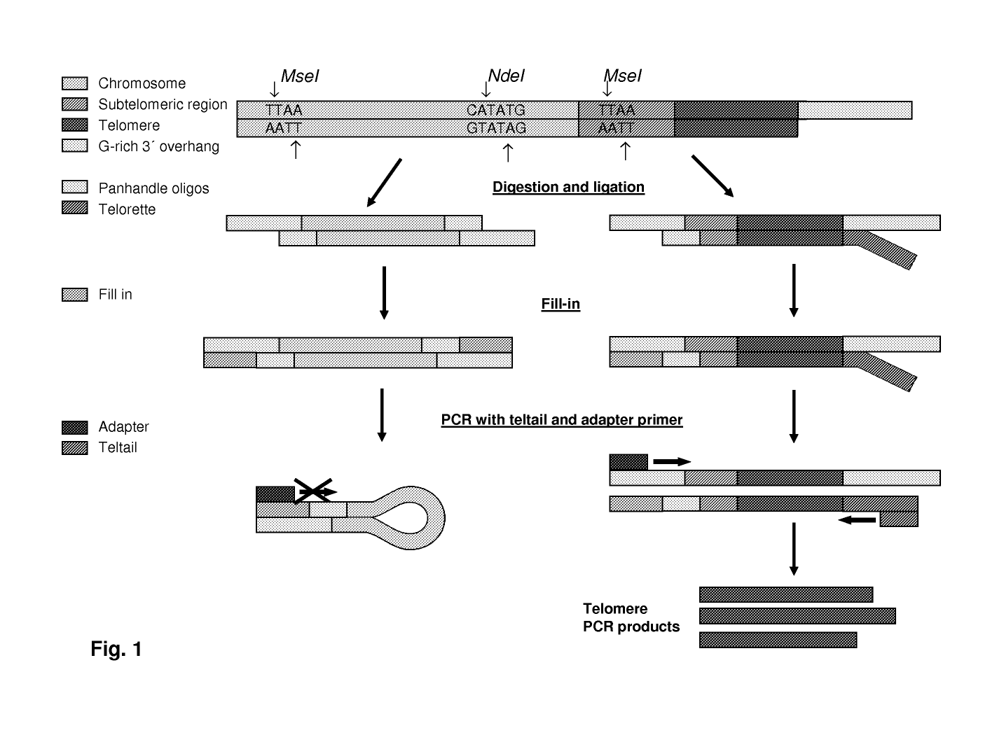

Suppression PCR has been described by others (Lavrentieva, 1999 and Broude, 2001) using panhandle oligos, which when ligated to both end of a DNA fragment, will guide the strands of the DNA fragment to self anneal during the PCR procedure and thereby preventing annealing of the primer and the following amplification of the DNA fragment. This requires that the double stranded region formed during self annealing has a melting temperature higher than the melting temperature of the primer employed. The sequences of the up-stream oligonucleotide applied in the examples of the present application are shown in Table 1. FIG. 1 illustrates the concept of suppression PCR showing the “panhandle” structure formed.

In a highly preferred embodiment the upstream oligo-nucleotide tag is a set of panhandle oligonucleotides.

It is clear to the person skilled in the art that sequences different to the sequence described herein can be used is this method.

Compared to the sequence used for panhandle PCR in the above cited reference (Broude, 2001) the sequence of the “short oligonucleotide” employed in the example herein has been extended by the addition of 5′-TA. This provides an overhang complementary to the overhang formed by the MseI/NdeI digest when the strands of the up-stream oligonucleotide tag are annealed. The end of the up-stream oligonucleotide tag should thus be constructed to be complementary to any overhang created by the digest of the genomic DNA as mentioned above.

The reasoning behind suppression PCR is that the long double stranded region formed by the GC rich double stranded region of the upstream oligo-nucleotide tag and the filling in reaction (se below) causes, when ligated to non-telomeric DNA fragments, formation of a secondary hairpin structure, which interferes with the subsequent PCR amplification and thereby inhibits amplification of non-telomeric DNA fragments.

This is especially pronounced if the melting temperature of this region is substantially higher than the melting temperature of the primers to be used in the amplification.

During the PCR procedure any excess of the upstream oligo-nucleotide tag, particularly of the short oligo can function as an extra primer, which can produce a very short fragment not containing telomere repeats. This process is counteracted by the short upstream oligo-nucleotide having a Tm, which is substantially lower than the Tm of the PCR primers. In this connection substantially lower is when the difference in Tm of the PCR primers and the short upstream oligo-nucleotide tag is such as more than 8° C. and not more than 40° C., preferably the difference in Tm is about, 10-30° C., such as about, 12-24° C. or most preferably about 14-22° C., such as about 16-18° C.

The Tm of the double stranded region of the upstream oligonucleotide tag is according to the invention above 20° C. and below 60° C., preferably the Tm of the double stranded region is 30-50° C., or more preferably 35-45° C., such as most preferably 38-42° C.

The upstream oligonucleotide tag should either directly or indirectly provide a primer binding site in the subsequent PCR amplification step and according to the invention a non-complementary sequence is preferred.

For the purpose of the application “a non-complementary sequence” is a nucleotide sequence which is not present in the telomeric or subtelomeric region, e.g. a sequence which is non-complementary to any sequence within the telomeric fragment to be amplified. It is thus preferred according to the invention that the up-stream oligonucleotide tag comprises a non-complementary sequence. Further preferred is the embodiment where the non-complementary sequence is a unique sequence, which is to mean that the unique sequence is non-complementary to any known genomic DNA sequence.

The region serving as primer binding site in the amplification step is preferably located out side the double stranded region of the up-stream oligonucleotide tag.

The upstream oligonucleotide tag according to the invention preferably comprises a single stranded region which encompasses said non-complementary sequence. The single stranded region is preferably such as more than 15 nucleotides long and less than 50 nucleotides long, more preferred are single stranded regions of about 20-40 nucleotides, such as more preferably about 25-35 nucleotides long.

The single stranded region, including the non-complementary sequence, is preferably comprised by the strand of the upstream oligonucleotide tag which is ligated to the G-rich strand of the telomeric fragments.

Using the examples described herein to illustrate the invention the subsequent PCR amplification is dependent on the filling in (see below) of the region opposite the single stranded region as the primer is identical in sequence to the non-complementary sequence. This set up is designed to optimise the subsequent PCR amplification.

In a highly preferred embodiment according to the invention the up-stream oligonucleotide tag comprises the pandhandle oligos identified by SEQ ID NO 1 and 2. In a more preferred embodiment the up-stream oligonucleotide tag is the pandhandle oligos identified by SEQ ID NO 1 and 2.

### Down Stream Oligonucleotide Tag

Previously a few methods employing ligation of an oligonucleotide to the downstream region of telomere fragments have been employed. The STELA method described in (Baird, 2003 and Sfeir, 2005) is used in the examples described herein with a few adaptations. Alternative methods which may be known to the skilled person may also be used.

The down-stream oligonucleotide tag is preferably ligated to the 5′end of the C-rich telomeric strand. This can according to the STELA method be guided by the presence of telomere complementary sequence. In higher organisms and particularly including all mammals and specifically humans the telomeric repeat is composed of the unit 5′TTAGGG-3, thus oligonucleotides comprising any representation of sequences complementary to this sequence may anneal to the G-rich strand and thereby be guided to the C-rich strand for ligation. The frame of the sequence may be shifted giving rise to different single stranded 3′ ends of the downstream oligonucleotide tag suitable for annealing to the G-rich strand of the telomere fragments.

The down-stream oligonucleotide tag is according to the invention preferably ligated to the C-rich strand of the telomere fragments.

According to the invention the downstream oligonucleotide tag preferably comprise a telomere complementary sequence of 4-15 nucleotides, such as 5-12, more preferably 6-10 or most preferably 7-9 nucleotides.

In an embodiment the telomere complementary sequence of the down stream oligonucleotide tag is located in the 3′ end of the downstream oligonucleotide tag and comprises an oligonucleotide sequence selected from the group consisting of SEQ ID NO 3-8. In a preferred embodiment the telomere complementary sequence of the down stream oligonucleotide tag is selected from the group consisting of SEQ ID NO 3-8.

The majority of telomere fragments are detected using oligonucleotide tags comprising 5′-CCTACC-3′, thus SEQ ID NO 5 is the preferred telomere complementary sequence.

The down stream oligonucleotide tag preferably comprises a sequence useful as primer binding sites for the subsequent PCR amplification. As described in connection with the up stream oligonucleotide tag the primer binding site is preferably a non-complementary sequence or more preferably a unique sequence.

The non-complementary/unique sequence is preferably located 5′ to the telomere complementary regions.

Said non-complementary/unique sequence is preferably more than 15 nucleotides long and less than 50 nucleotides long, more preferred 15-40 nucleotides, such as most preferably 15-25 nucleotides long.

The total length of the down stream oligonucleotide tag is preferable 18-70 nucleotides, such as preferably 20-40, such as 25-30 nucleotides

It is clear that the primer binding sites, e.g. the non-complementary sequence or unique sequence of the up and down-stream oligonucleotide tags should not be identical.

In an embodiment the downstream oligonucleotide sequence is selected from the group consisting of the sequences identified by SEQ ID NO 9-14.

As described herein (se above) the telomere complementary sequence identified by SEQ ID NO 5 detects approximately 80% of the telomere fragments detected using such protocols. Thus the reactions using the remaining of the sequences can be omitted in several procedures. The down stream oligonucleotide tag identified by SEQ ID NO 11, including SEQ ID NO 5, is highly preferred.

### Over all Procedure

The steps b and c of the method according to the invention may be performed in any order, or simultaneously.

In preferred embodiments of the invention both the up and down stream oligonucleotide tags are constructed to comprise sequence complementary to the telomere fragments generated by the initial digestion step. Therefore the preferred method may include annealing of either of the oligonucleotides.

In preferred embodiment the method according to the invention, comprising the following steps:
a) digestion of a genomic DNA preparation generating telomere fragments
b) annealing of an up-stream oligonucleotide tag to the telomere fragments and ligation of the up-stream oligonucleotide tag to the telomere fragments
c) annealing of a down-stream oligonucleotide tag to the telomer fragments and ligation of the down-stream oligonucleotide tag to the telomere fragments,
d) amplification of telomere fragments using primers with a sequence complementary or identical to at least part of the up- and downstream oligonucleotide tags and
e) estimate telomere length by determining the length of the PCR amplified telomere fragments.

In the preferred embodiment as outlined herein above both of the oligonucleotide tags, comprise regions facilitating ligation to the respective ends of the telomere fragments. The olignucleotide tags comprise regions complementary to either ends of the telomere fragments, e.g. the up-stream oligonucleotide tag may as in the example described herein have an overhang complementary to the overhang created by the enzymatic digestion of the chromosomal DNA and the down-stream oligonucleotide may comprise a telomere complementary region.

The method may accordingly include annealing of each oligonucleotide tag to the telomere fragments prior to ligation of each tag.

The conditions for the steps b and c are to be suitable for ligation and possible annealing. The conditions may be changed to optimise the individual steps and sub-steps. A ligation reaction requires an energy input, preferably in the form of ATP, which is to be included in the reaction buffer. Ligase enzyme is commercially available and may be used according to the manufacture. Annealing requires a suitable buffer and temperature allowing hybridization of the complementary regions. Conditions that affect hybridization efficiency are further described in the definitions.

In order to minimize the handling of the samples, e.g. the number of intermediate purification steps a procedure has been developed allowing the method to be carried out as a one step method without any extraction and precipitation steps.

As described in the examples here in, the annealing of step b is preferably performed while lowering the temperature from 65° C. to 16° C. over and hour. The subsequent ligation is preferably performed at 16° C.

Step c is preferably performed at a higher temperature as the down stream oligonucleotide tag has a longer stretch of nucleotides (the telomere complementary region) annealing with the 3′ overhang of the telomere fragments. The reaction may according to the invention be performed at any suitable temperature, such as above 10° C., such as above 15° C., such as above 20° C., such as preferably above 30° C. The annealing is preferably performed below 50° C., such as below 45° C., such as more preferably below 40° C. Most preferably the annealing and ligation of the down stream oligonucleotide tag is performed at 30-40° C., such as at 32-38° C. an most preferably at 34-36° C.

The duration of the annealing and ligation step b and c may be such as 2 hour, such as 3 hours, such as 8 hours, such as 12 hours, such as 18 hours. The annealing and ligation step may conveniently be performed over night (ON) that is such as at least 8 hours and maximum 24 hours, most preferably such as 12-18 hours.

The buffer compositions used may be any suitable buffer, such as NEW buffers, preferably NEB 2 with the addition of ATP.

As noted above the annealing and ligation may be performed using different conditions reflecting the nature of the oligonucleotide tags used. The person skilled in the art will understand how to vary the conditions depending on the precise sequence of the oligonucleotide tags used in the method. General methods employed in molecular biology may be found in textbooks related to molecular biology.

Preferably an inactivation step, such as heating to 65° C. for 20 minutes is, applied prior to the amplification step.

### Fill in

As described above in relation to the description of the up-stream oligonucleotide a preferred embodiment of the invention employs an upstream oligonucleotide tag, which is partially double stranded and partially single stranded. Following annealing and ligation of this oligonucleotide tag a single stranded overhang is present in the upstream end of the telomere fragment. This structure of the olignucleotide tag optimizes the PCR reaction, as filling in of this region is needed for the subsequent binding of the upstream primer.

In an embodiment step b give rise to an overhang. In a preferred embodiment this single stranded region is rendered double stranded by including a filling in step, which is a polymerase reaction which may be performed by any method know to the person skilled in the art. In a preferred embodiment the method according to the invention includes a step of filling in.

In a preferred embodiment the filling in is performed as an initially step of the PCR reaction, which may be feasible if a polymerase which is not a hotstart enzyme is used, whereby an elongation step can be performed prior to the amplification cycles (se below).

### Up- and Down Stream Primers.

The method according to the invention comprises an amplification step, whereby the telomere comprising fragments are amplified, preferably using the polymerase chain reaction (PCR). Amplification of the telomere fragments enables detection of telomere fragments starting from as low quantity of starting material—e.g. chromosomal DNA (see below).

As described above the up- and down stream oligonucleotide tags are according to the invention constructed to provide both up and down stream primer binding sites, with sufficient specificity.

In a preferred embodiment the up stream primer comprise a sequence identical or complementary to at least part of the non-complementary sequence or unique sequence of the up stream oligonucleotide tag. In the examples described herein a preferred up stream primer of SEQ ID NO 15 is used.

In a preferred embodiment the down stream primer comprise a sequence identical or complementary to at least part of the non-complementary sequence or unique sequence of the down stream oligonucleotide tag. In the examples described herein a preferred down-stream primer of SEQ ID NO 16 is used.

It is clear that the sequences of the up- and down stream primers identical or complementary to at least part of the non-complementary sequence of the up- and down stream oligonucleotide tags must be sufficiently different to avoid cross hybridization.

The primers according to the invention preferably have a Tm of 40-80° C., such as 50-70° C., such as 55-65° C., such as 58-64° C., or more preferably 60-64° C., or most preferably 62-64° C.

The primers according to the invention preferably have a length of 10-30 nucleotides, such as 15-25, such as preferred 17-24 nucleotides, such as more preferred 18-23 nucleotides, such as most preferred 19-22 nucleotides

It is clear to person skilled in the art that the precise sequences of primers can be altered.

The primers are oligonucleotides e.g. short sequences of nucleotides which are conveniently prepared by an automated synthesizer. Oligonucleotides can be prepared by using any nucleotide available that is the nucleotides of DNA and RNA (dATP, dTTP, dGTP, dCTP, ATP, UTP, GTP and CTP). Alternatively non-natural nucleotide may also be used according to the invention, such as nucleotide derivatives. Nucleotide may be labeled by use of any suitable label such as enzymes, chromophores, radioactive tracers and fluorophores may be linked to the nucleotides. Preferred are fluorescent dyes, which are used for multiple purposes in molecular biology such as DNA sequencing.

Preferred labels are FAM or HEX or other fluorophores that can be visualized on capillary electrophoresis equipment.

Such labelling can according to the invention be incorporated in the up- and/or down-stream primers to facilitate detection of the amplification product. Alternatively a fraction of nucleotide used in the amplification reaction can be labelled.

In a preferred embodiment the up and/or down stream primers are/is labelled.

### PCR Reaction

In a preferred embodiment the amplification is performed by the polymerase chain reaction (PCR). The condition for the PCR amplification may be adapted by the person skilled in the art taking into account the enzyme(s) to be used, and the structure of the primers to be used.

As described in the examples herein an amplification cycle adapted to the primers described herein has been developed.

The PCR reaction requires suitable enzyme(s), buffers, nucleotides and the selected primers.

As described above a fill in step may be required prior to the amplification. This may as described before be performed as an initial step of the PCR reaction.

### Genomic DNA Preparation

One object of the present invention is a method for estimating telomere length which can be performed using a low amount of starting material. In multiple situations where information of telomere length is desired, the availability of genomic DNA may be a limiting factor when employing methods such as TRF, wherein the telomere fragments are not amplified.

The genomic DNA used in the method according to the invention may be prepared using any suitable method known in the art. Such methods are known by the skilled person. Several commercially available kits are available and may be used according the manufactures instructions.

The method according to the invention is preferably performed using 5 pg-1 ng ligated DNA, preferred is such as 10-500 pg, more preferred such as 20-100 pg or most preferred is such as 20-40 pg ligated DNA pr PCR reaction.

The result of the amplification method according to the invention is highly variable from sample to sample and thus in order to have a reliable estimate of telomere length multiple reactions are performed using different samples of the same digested and ligated DNA preparation. The data presented in the examples includes 4 to 8 lanes per digested DNA sample and the results are thus based on the average of data obtained.

The specific reaction conditions described in the example has been developed to minimize handling of the samples, by removing the necessity of intermediate precipitation or purification steps. In a preferred embodiment the steps b)-d can be performed with out intermediate precipitation or purification steps. It is like-wise preferred that the steps a)-c) can be performed in a one-tube system.

It is further possible according to the invention that the PCR amplification products from at given digested DNA sample can be pooled prior to analysis, whereby the number of samples to be analysed can be decreased. In an embodiment the amplification products obtained by step d) are pooled before performing step e).

### Determining the Length of PCR Amplification Products

The length of the PCR amplification products are according to the invention detected using any suitable method known in the art.

As described in the examples herein the PCR products may be separated by gel electrophoresis on a 0.8% TAE Seakem agarose gel (run at low voltage over night for better separation of the distinct bands), and transferred to a nylon membrane by Southern blotting using a vacuum blotter. The blotted DNA fragments can then be hybridized overnight to a DIG (digoxigenin)-labeled probe specific for the telomeric sequence and subsequently incubated with a DIG-specific antibody coupled to alkaline phosphate. Finally, the telomere probe may be visualized using a chemiluminescent substrate (CDP-Star) and the chemiluminescence signal can be detected using a BioImager from UVP. The lengths of the amplified bands can be calculated using Vision Works software from UVP.

The probe is preferably telomere or subtelomere specific, most preferably telomere specific. Such probes are know in the art and the use of such probes can be employed by the skilled person using guidance in the prior art.

It is clear that the detection of the PCR products can be performed using any suitable detection technique. The probe may be detected using any suitable labeling and detection system and following be analyzed using any suitable software.

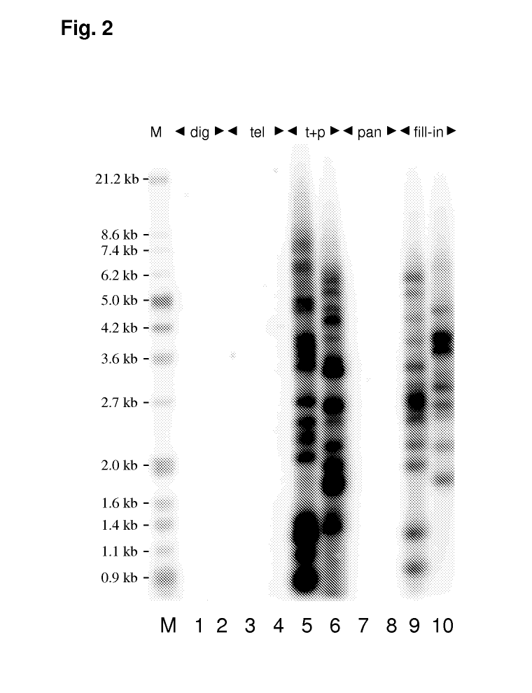

The data obtained from the method according to the invention may be depicted by autographics or scannings as shown in FIGS. 2-7. The results are further discussed in the example.

In a preferred embodiment the PCR amplification product are labeled by incorporation of labeled primers or labeled oligonucleotides. The preferred label is a fluorescence label. If labeling of the PCR amplification product is employed the length of the products may be determined using the incorporated labels. In a preferred embodiment an automatic system, such as capillary electrophoresis may be used.

### A Kit of Parts

An aspect of the present invention relates to a kit of parts comprising two or more components for carrying out one or more steps of the method according to the invention said kit comprising:
a) restriction enzyme(s) and/or
b) one or more oligonucleotide tags selected from up and/or down-stream oligonucleotide tags and/or
c) one or more primers selected from up and/or down-stream primers and/or
d) ligase and/or
e) ATP and/or
f) components for PCR (buffers, NTPs, polymerase) and/or
g) hybridization probe
h) instructions for carrying out the method.

Each component of the kit of parts may be defined as described herein.

A further aspect of the invention relates to the downstream oligonucleotide tag as described by SEQ ID NO 1 and SEQ ID NO 2.

The invention further describes the use of the kit as mentioned above, the oligonucleotide as mentioned above and/or the primer identified by SEQ ID NO16 in a method for estimating telomere length, particularly in a method as described herein.

### Application of the Method According to the Invention

The information obtained using the method according to the invention (as discussed in the example), is not an accurate determination of the exact telomere length. The method is due to the procedure biased towards detection of the shortest telomere fragments, which is for the most parts also considered the most interesting telomere fragments, as the shortest telomeres are the ones which may first lead to loss of genetic information if the remainder of the telomere is lost during cell divisions.

The method may be used for estimating telomere length in a biological sample, said sample may as described previously be such as a biopsy sample, blood sample, buccal swap, faecal sample or any other suitable sample capable of providing sufficient DNA material. The sample is preferably a blood sample which comprises lymphocytes, wherefrom genomic DNA may be extracted. In specific situation, depending on the purpose of the analysis wherein the method is employed tissue or cell samples may be used as appropriate.

The knowledge of telomere length or mean length of the shortest telomers may be used in numerous situations. In an embodiment the method according to the invention is for use in assessing telomere dynamics, which may be relevant in many situations, such as in the connection with aging. In a further embodiment the method according to the invention is for use in assessing the effect of modulation of telomerase activity.

As telomerase and telomere length are associated with senescence the method according to the invention may be for use in assessing remaining proliferative capacity or lifespan. The method may be used in a diagnostic method.

Due to the findings that telomerase and telomere length as described in the background section appears to play a role in cancer development the length of telomeres is a highly interesting feature in the field of cancer diagnostics, prognostic and therapeutic methods.

In an embodiment the method according to the invention is for use in a diagnostic, prognostic and/or therapeutic method, and in a preferred embodiment the method is for used in a diagnostic, prognostic and/or therapeutic method of cancer.

In this connection an estimate of telomere length may be used to evaluate the applicability of a specific treatment by giving an estimate of the proliferative capacity of the cells, the method may be used to assess the tolerance of cells towards cytotoxic treatments such as radiation therapy. By using such methods an individualized treatment which is suitable for the individual patients can be applied.

In a further embodiment the method according to the invention is for use in assessing a potential anti-cancer treatment and/or in another cancer related procedure.

There are a plurality of disease where the impact of telomere length has been investigated, such as hypertension (Benetos, 2004), infections (Cawthon, 2003), arthritis such as osteoarthritis or degenerative joint disease. The overall conclusion is that the shorter telomere the higher is the risk of a disease. Smoking has also been found to result in shorter telomers.

It appears that the cumulative effect of stress and wear throughout an individuals life manifest it self as short telomeres. Thus an estimate of telomere lengths may give an indication of the over all heath state of an individual.

In a specific embodiment the method according to the invention, for use in assessing the stability of donor stem cells in bone marrow transplantation.

In a different embodiment the method according to the invention is for use in assessing, treating or diagnosing male infertility.

### Example

The following examples are to illustrate the method according to the invention and are not to be interpreted as limiting for the invention.

### Overview of Assay Principles

Extracted DNA from as little as 250 cells is digested with a mix of the restriction enzymes here MseI and NdeI, which produce two-base sticky overhangs and are frequent cutters presumably also cutting the subtelomeric region (see FIG. 1). The digestion leaves behind mainly pieces of genomic DNA of 10-3000 bp all with the same sticky overhang 5″-AT-3′ in each end. However for every chromosome end a fragment of DNA with the telomeric region including the 3′ overhang and a smaller part of the subtelomeric region with a 5″-AT-3′ overhang is also formed.

The next step is a ligation-based step, in which two specially designed oligonucleotides are ligated to the upstream overhang. These two oligonucleotides are designed so that they anneal forming a two-base sticky overhang complementary to the overhang formed by the digestion. The other end of the oligo pair is long, single-stranded and GC-rich overhang. The annealed oligonucleotide complex is termed “upstream oligonucleotide tag”. An oligonucleotide tag is exemplified by the 11+2-mer (SEQ ID NO 1) and the 42-mer (SEQ ID NO 2) shown in table 1.

The third step is another ligation step wherein a down stream oligonucleotide tag (telorette in STELA) is annealed to the G-rich 3″-overhang of the telomeric repeat. This oligo consists of seven bases complementary to the telomere and a tail of 20 non-complementary nucleotides. After annealing the telorette is ligated to the 5″-end of the C-rich strand of the telomere. The sequences of telorette 1-6 are identified by SEQ ID NO 6-14 (table 1).

A fill-in step is required so that the GC-rich upstream overhang of the upstream oligonucleotide tag becomes double stranded and hereby capable of serving as template for the upstream PCR primer (se below).

In the first step of the PCR reaction all the DNA pieces are denatured. When the temperature is again lowered for the annealing step two things can happen. For the telomeric fragments (see FIG. 1 right side) the upstream primer (SEQ ID NO 16, table 1) will anneal to the filled-in part of the upstream sequence thereby initiating a PCR reaction copying also the downstream oligonucleotide tag. In the following PCR cycles the down stream primer (teltail primer in STELA) will be able to anneal to the PCR product obtained by the upstream primer, thereby producing PCR amplification products of different lengths reflecting the lengths of the individual telomeres including any subtelomeric DNA present with in the original telomere comprising DNA fragments.

For the intra-genomic fragments where the upstream oligonucleotide tag is ligated to both ends, the complementary ends will anneal to each other, forming a pan-handle, which will be relatively stable due to a higher melting temperature of the panhandle sequence. The PCR reaction, based on intra-genomic fragments as template, will therefore be suppressed (see FIG. 1 left side).

The whole procedure can be done in a one-tube system and with no intermediate precipitation or purification steps. By minimizing handling and loss of DNA the method can be applied to large series of samples. The method further requires only very small amounts of starting material.

### Material:

Cell culture: We obtained fibroblast strains WI-38 and WI38 VA13 subline 2RA and the cancer cell lines HeLa and NC1-H1299 from ATCC. DNA from cancer cell lines HL60 and U937 were purchased from Roche Applied Bioscience. Two strains of hTERT immortalized human mesenchymal stem cells were kindly provided by N. Serakinci, IMB, University of Southern Denmark. A cancer cell line MCF7 was kindly provided by A. E. Lykkesfeldt, Dept. of Tumor Endocrinology, Danish Cancer Society.

We obtained blood samples from four healthy, fully informed volunteers and from four old persons from the Longitudinal Study of Aging Danish Twins (Bischoff, 2004).

DNA from cell cultures was extracted using the Master Pure Purification kit from Epicentre while DNA from blood samples were extracted using the common desalting procedure.

In order to validate the procedure results were compared to results obtained by the TRF assay and by XpYp STELA.

### TRF Assay

For TRF assay-based determination of telomere length the TeloTAGGG telomere length assay from Roche was used according to manufactures manual with few adaptations. In principle 0.5-1 μg of isolated DNA was digested by either HinfI/RsaI (Roche), MseI/NdeI or HphI/MnlI (from NEB). The HinfI/RsaI mix is known to cut outside the subtelomeric region while HphI and MnlI recognize the telomere repeat variants TGAGGG and TCAGGG respectively.

The digested DNA was separated by gel electrophoresis on a 0.8% TAE Seakem agarose gel, and transferred to a nylon membrane by Southern blotting using a vacuum blotter. The blotted DNA fragments were then hybridized overnight to a DIG (digoxigenin)-labeled probe specific for the telomeric sequence and subsequently incubated with a DIG-specific antibody coupled to alkaline phosphate. Finally, the telomere probe was visualized using a chemiluminescent substrate (CDP-Star) and the chemiluminescence signal was detected using a BioImager from UVP. The TRF lengths were calculated using Vision Works software from UVP.

### XpYp STELA

XpYp STELA was adapted from Sfeir et al. Isolated DNA was digested by EcoRl, quantified by Picogreen (Molecular Probes) and diluted if necessary. To 10 ng of digested DNA, 20 U of T4DNA ligase, 10⁻³ μM telorette, 1× NEBuffer2 and 1×ATP was added in a 15 μl volume and left overnight at 35° C. followed by a 20 min inactivation step of 65° C. DNA was diluted to 250 pg/μl with water.

Multiple PCR reactions was carried out for each sample in a 12 μl volume containing 200-500 pg of ligated DNA, 1× Failsafe PCR PreMix H (Epicentre), 0.1 μM teltail and XpYpE2 primers and 1.25 U of Failsafe Enzyme (Epicentre). The reaction was carried out on a Hybaid Thermocycler (Thermo Electron) under the following conditions: 1 cycle of 95° C. for 2 min, 26 cycles of 95° C. for 15 s, 58° C. for 30 s and 72° C. for 10 min, 1 cycle of 72° C. for 15 min. (For oligo and primer sequences see table 1)

Detection of the PCR products was done as described for the TRF assay with the exception that the agarose gel was run at low voltage over night for better separation of the distinct bands. The size of the PCR products was calculated on basis of the molecular weight marker (Roche) using VisionWorks Software from UVP (results not shown).

### Detailed Example of a Method According to the Invention

Isolated DNA was digested by 1:1 mixture of MseI and NdeI, quantified by Picogreen (Molecular Probes) and diluted if necessary. 10 ng of digested DNA is mixed with 50 μmol 42-mer and 50 μmol 11+2-mer in a 7 μl volume. (For oligo and primer sequences see table 1) The mixture was ramped down from 65° C. to 16° C. over 1 hour. 20 U T4 DNA ligase (NEB) was then quickly added together with 1× NEBuffer2 and 1×ATP and left overnight at 16° C. Additionally 20 U of T4DNA ligase and 10⁻³ μM telorette was added and the reaction mixture was supplemented to 1× NEBuffer2 and 1×ATP in a 25 μl volume and left overnight at 35° C. followed by a 20 min inactivation step of 65° C.

PCR reactions was done in a 12 μl volume containing 20-50 pg of ligated DNA, 1× Failsafe PCR PreMix H (Epicentre), 0.1 μM teltail and Adapter primers and 1.25 U of Failsafe Enzyme (Epicentre). The reaction was carried out on a Hybaid Thermocycler (Thermo Electron) under the following conditions: 1 cycle of 68° C. for 5 min, 1 cycle of 95° C. for 2 min, 26 cycles of 95° C. for 15 s, 58° C. for 30 s and 72° C. for 12 min, 1 cycle of 72° C. for 15 min. The initial step of 68C was the fill-in step. This step can also be done separately prior to the PCR reaction. This was done in the same tube as the ligations in a mix of 1× AmpliTaq Buffer (Applied Biosystems), 1 mM MgCl, 0.2 mM dNTP and 1 U AmpliTaq enzyme (Applied Biosystems) in a 50 μl volume.

Detection of telomere repeat fragments was done as described for XpYp STELA.

### Results

### Method Development and Validation

The method according to the invention was developed by optimizing each step of the procedure separately. The end point of these optimizations was always the visualization of telomere-containing PCR-products, using the Southern blot technique.

As is also seen with the chromosome-specific STELA, the PCR products are seen as a multitude of discrete bands, where each band undoubtedly represents the length of one single telomere block from the DNA sample used as template. Due to the special mechanism of maintaining telomeres, it must, however, be expected, that even very small samples of DNA will contain telomere blocks of many different lengths. A consequence of this is, as also clearly demonstrated by Baird et al, 2003, the appearance of different band patterns even when analyzing different samples of the same primary DNA preparation. The following optimization steps were therefore evaluated based on length and intensity of bands without placing much emphasis on the fact that the detailed banding pattern could be different between experiments.

### Digestion

The digestion was carried out with a mix of two restriction enzymes leaving the same sticky overhang. It has previously been shown by others (Baird et al, 2006) that the TRF assay is sensitive to the restriction enzymes used, therefore we initially did a TRF assay with three different mixes of restriction enzymes: HinfI/RsaI, MseI/NdeI and MnlI/HphI. By doing so we found that the TRF assay produced significantly shorter (˜0.8 kb) fragments using the MseI/NdeI mix than the original HinfI/RsaI mix and only slightly longer (˜0.4 kb) fragments than when using the MnlI/HphI mix. This suggests that using the MseI/NdeI mix we cut the DNA close to the telomeric repeats and most likely in the subtelomeric region, thereby partly overcoming one of the problems with the TRF assay wherefore the MseI/NdeI mix was chosen for the further studies. This also suggests that we have a very short, although unknown, part of non-telomeric DNA in our final PCR product.

### Ligation

The first ligation step was documented to be successful since this step is necessary in order to produce a PCR product (see FIG. 2). As mentioned above we designed the “panhandle oligos” to form a panhandle that should quench the production of PCR-fragments formed by restriction fragments with panhandle sequences ligated to both ends. When using large amounts of DNA we did, however, observe a smear of shorter bands in an agarose gel stained with ethidium bromide but not on the blot with the telomeric specific probe. This suggests amplification of some non-telomere associated fragments in spite of the panhandle, a fact that support the notion that ligation of the panhandle oligo does occur.

We have furthermore modified the ligation of the down stream oligonucleotide tag (telorette) compared to Sfeir et al (Sfeir, 2005) by using a different buffer and by not extracting the DNA in between steps (data not shown).

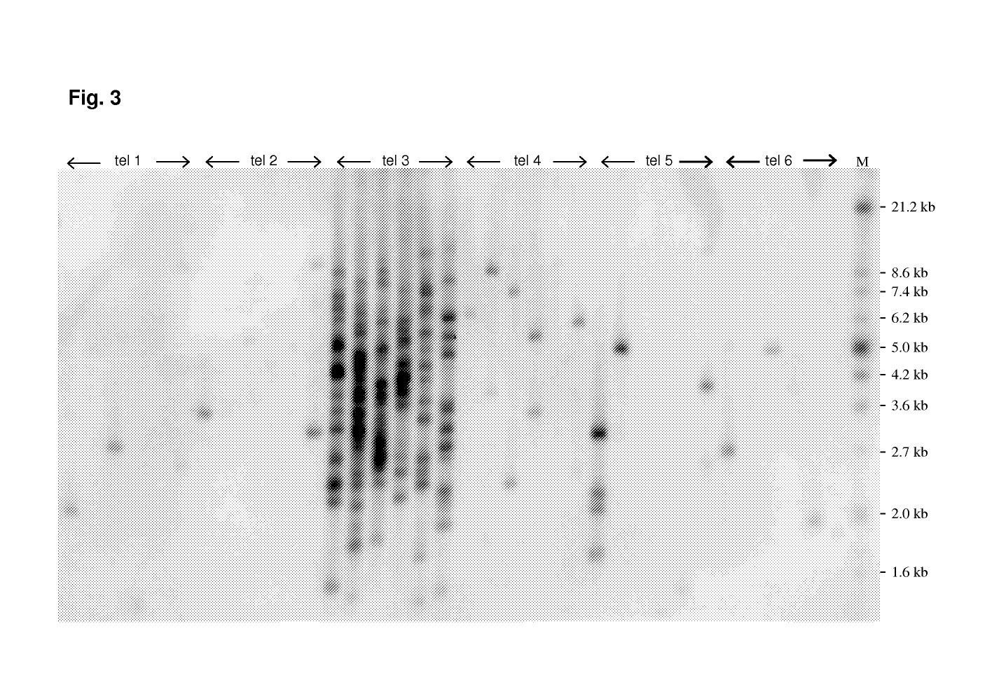

It has earlier been shown by Sfeir et al that the last nucleotide of the 5′ end is CAATCC at 80% of the chromosome ends. The method according to the invention gives the same result (See FIG. 3).

### Fill-in

The fill-in reaction was initially included as a separate step. But in further developing the method we have successfully exploited the fact that the Failsafe enzyme is not a hotstart enzyme, and therefore we have build in a cycle at 68° C. as the first step in the PCR to fill in the gap of the upstream oligonucleotide tag. In FIG. 2 we demonstrate that we obtain products when carrying out the fill-in as a separate step as well as when including it as a part of the PCR cycling. The figure also shows that omission of the fill-in step tends to give slightly longer products than when including a separate fill-in step. This is probably due to the fact that a separate fill-in step means a slight change in buffer composition at this point.

### PCR

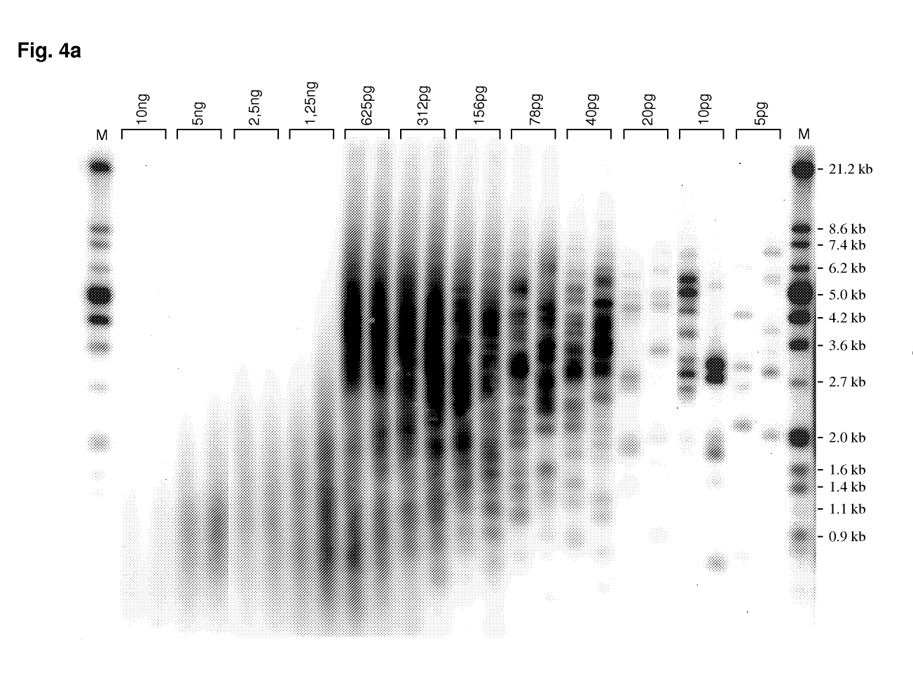

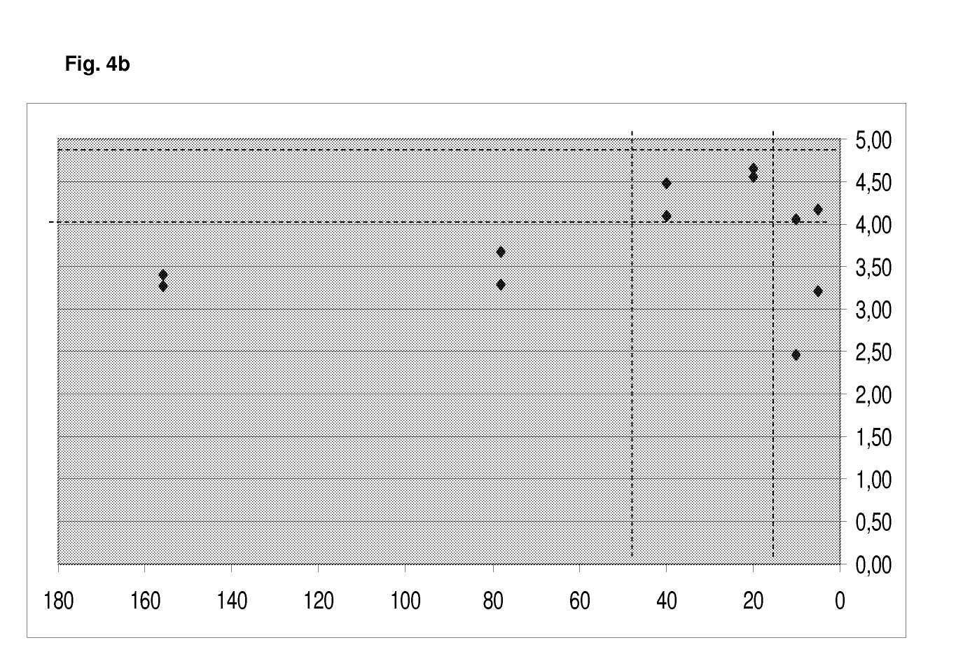

The PCR reaction has been validated on DNA from different cell type. Due to the special nature of telomere maintenance no telomeres are expected to be of exactly the same length. Therefore we chose to run 8-15 different PCR reaction for each sample as also done by others using the ordinary STELA (Baird, 2003 and Sfeir, 2005). The PCR reaction needs very small amounts of template DNA. This is one of the major advantages of this method. But it is also an aspect of which one has to pay great attention. In FIG. 4 we show how descending concentrations of template influences the PCR products.

### Estimation of Mean Telomere Length

As shown in FIG. 4a the banding pattern achieved with the method according to the invention depend strongly on the amount of template DNA. The general trend is, however, that discrete bands occur only when the amount of template DNA is below 200 pg. We chose to use this limit and only include data obtained from PCR reactions with less than this amount of template in the following. As an estimate of mean telomere length we used the mean of the estimated length of all individual bands in the lanes. When estimating the mean telomere length in this way and performing multiple measurements on the same sample we obtained day-to-day coefficients of variation in the range 0.03. FIG. 4b depicts estimated mean telomere length values obtained this way for template DNA amounts in the range 5-156 pg pr assay. We consider template amounts between 15-45 pg per assay as optimal. Amounts above 45 pg results in underestimation, while amounts below 15 pg results in very few bands and therefore in a high imprecision in the estimation of the mean.

### Validation

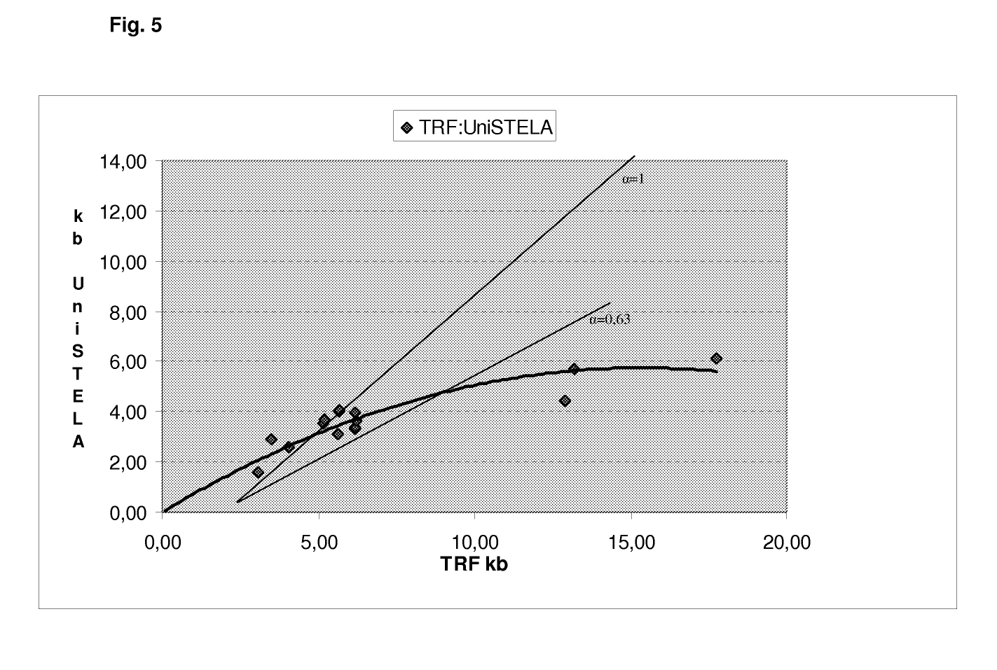

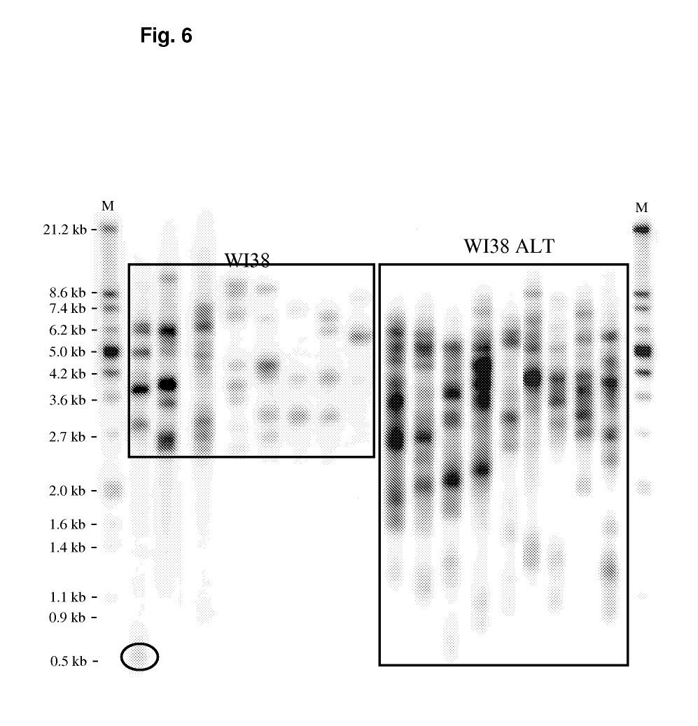

FIG. 5 shows the relationship between mean telomere length estimates obtained by the regular TRF assay and by the method according to the invention, calculated as described above. The TRF assays give consistently longer estimates than the method according to the invention measurements. This was expected since the method according to the invention favors the shorter telomeres due to the limitations of PCR while the TRF has difficulties picking up the shorter telomeres thereby overestimating the length. From the curve in FIG. 6 we see that the curve comes close to a linear fit up until 8 kb. When analyzing samples with TRF lengths below 8 Kb we find a close to linear correlation between the two assays (y=0.513x+0.637; R²=0.64). As for samples with very long TRF lengths (14-20 kb) the curve is almost horizontal, illustrating the limitation of our method in producing PCR products from very long templates.

### Biological Application

We have as examples of biological applications applied the method to the fibroblast strain WI38 and the ALT positive daughter line WI38 VA13 subline 2RA. The results are depicted in FIG. 6. One striking finding that is clearly illustrated in FIG. 6 is the pronounced diversity in telomere length in ALT cells compared to non-ALT cells.

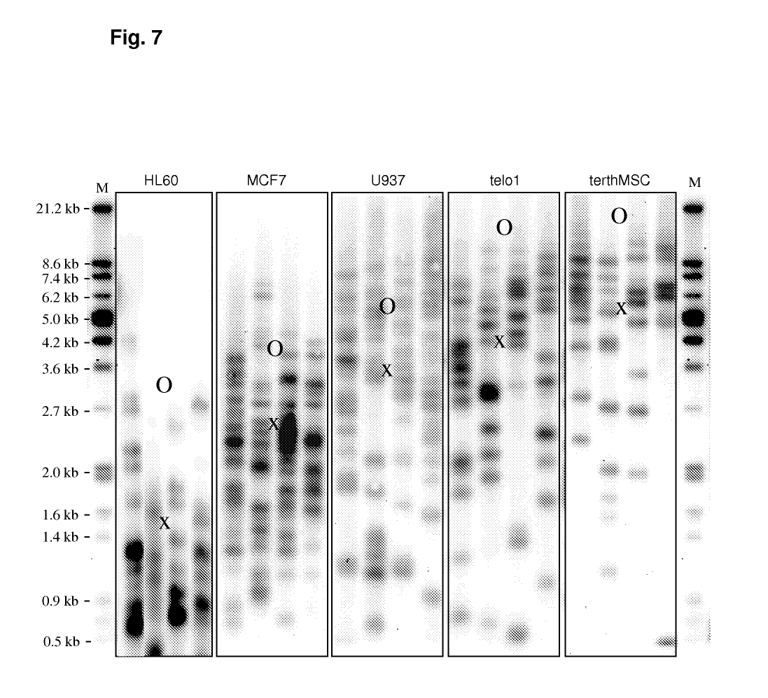

We have also analyzed a series of telomerase-positive cells samples with variable telomere lengths. The results are shown in FIG. 7. In this series the striking finding is that although the mean telomere length is increasing towards the right side of the figure, all cell samples have a subpopulation of very short telomeres that would be missed by traditional TRF-assays.

### Discussion

Above is described the development of a new method for measurement of telomere length. This method has one important advantage compared to TRF-assays, namely that it is based on PCR-technology, which means that analysis can be performed on minute amounts of sample.

Two PCR-based methods for telomere length measurements previously presented both have limitations. The method published by Cawthon (Cawthon, 2002) is a method where it is not the length of the telomere repeat block that is measured, but instead the amount of telomere repeat sequences in a DNA sample, that is quantified. In principle this should be as precise as length measurements, if measurements of non-variable reference sequences are included, which Cawthon has done. Difficulties of the method include the rather trivial problem, namely that measurements in the Cawthon assay requires precise pipetting of template DNA, which is notoriously difficult. The other problem relates to the kinetics of the PCR reaction, which is rather unpredictable, resulting in an extreme sensitivity to template amounts and very varying slopes when plotting product again PCR cycles. We are of the opinion that most of these problems stem from the fact that in the Cawthon reaction non-full length products can serve as additional primers, making the whole reaction difficult to control.

The other PCR based method for telomere length measurements is the original STELA method, where the PCR reaction is performed between a primer sequence ligated to the 3′-overhang of the telomere repeat and a chromosome-specific upstream sequence. By the development of the present method the main disadvantage of the original STELA method has been circumvented, namely that it measures telomere length on only the few chromosome ends, where a chromosome-specific, telomere-near, unique sequence could be found. This prerequisite is at present only fulfilled for Xp, Yp, 2p, 11q, 12q and 17p. We therefore set out to develop a method where STELA-like PCR could be run on all chromosome ends, a goal that we achieved by establishing a method where a restriction site upstream of the telomere block were used to ligate another primer binding site useful for PCR.

In overcoming the disadvantage of the original STELA we loose the information on which chromosome arm the shortest telomeres are located to. We consider it less significant to know what chromosome arm is the shortest, but of absolute importance to know the distribution of the shortest telomeres.

In the development of the method special attention was paid to two aspects. Firstly we felt that there could be a risk that the production of telomere-containing fragments would be hampered by the vast majority of intra-genomic template fragments, that all had the upstream primer sequence ligated to both ends. We therefore included the panhandle concept in the design, in order to minimize this non-telomere related PCR reaction. The suppression of the nontelomere PCR was evaluated by comparing the nontelomeric smear at different amounts of template. The non-telomeric smear can—apart from the fact that it does not stain with telomere-probes—be recognized by the fact that it is much shorter than the telomere fragments. We found that when using the panhandle approach the amounts of non-telomeric PCR product is modest, as long as the total template amount is below 100 pg. The possible interference of remaining, small amounts of non-telomeric PCR products are minimized by the fact that we chose to visualize the telomere fragments by Southern blotting, using a telomere-specific probe.

The other aspect, that we have paid special attention to, is to make the reaction as simple as possible. In the development of the method we started out with performing all steps separately, but after having established the method we focused on simplifying the method as much as possible in order to make it suitable for large-scale series. We have had substantial success with this, resulting in a method, that can be performed without purification steps and with only a minimum of transfers of the reaction mixture from tube to tube. Another advantage of the method is that it performs on very small amounts of DNA. As seen in FIG. 4a the method performs best on template amounts in the range 15-45 pg ligated DNA, which corresponds to the DNA from only a few cells. With this amount of template we achieve a reasonable number of clearly separated bands, where the length of individual bands can be measured. With less than 15 pg the number of bands starts to be too few for reliable estimates and at amounts over 45 pg bands starts to merge, making measurements problematic. At very high amounts of template, one extra problem is that the smear from intra-genomic fragments starts to give a certain background stain. The question can be raised why 15-45 pg DNA gives so few fragments compared to the expected number of telomere-containing fragments expected in such an amount of DNA. We do not believe that the reason is low efficiency of the ligation reactions, mainly because different samples of the same ligation reaction give different fragments, suggesting that many different telomere-containing fragments are present in the reaction at the start of the PCR. In stead we assume that the PCR reaction may be delicate, resulting in only a limited number of fragments starting amplification from the first PCR cycle. It is assume that these relatively few template fragments, that by a purely stochastic process are starting to amplify in early cycles, are the ones we see in a lane. Such a stochastic element would also explain why the pattern of bands is different from lane to lane, even though the template DNA in all lanes comes from the same ligation reaction.

With regards to validation of the method, we have chosen to do this both by determining between-day variation in estimating mean telomere length and by performing a comparison with results obtained by the TRF-assay. We therefore initially had to choose by which method to extract a mean telomere estimate from our data. We have found that the highest precision and best correlation with data from TRF-assays were obtained when using the following procedure to obtain a mean telomere length. After running nine separate PCR reactions on the same ligation mixture the sizes of all single fragments are calculated on basis of the molecular weight marker using appropriate software, without correcting for differences in intensity of bands and then calculating a mean of these individual length estimates. In this way we obtained acceptable estimates of precision (between-days CV: 0.03) and a close to linear relationship to corresponding TRF-values, as long as the mean telomere length value was under 8 Kb. Above this length it is obvious that the PCR-reaction starts to suffer.

A consequence of this is that the method according to the invention does not give a precise estimate of the mean telomere length of a sample with very long telomeres. We are, however, of the opinion that this fact is of lesser significance since we believe that the essence of the telomere dynamics lie in the distribution of the short telomeres and not in a mean length.

A striking fact apparent in FIG. 4b is that even for mean telomere length estimates below 8 Kb the values achieved using the method according to the invention is significantly shorter than the estimates achieved by TRF-assays. The explanation for this is probably due to a combination of limitations to the two assays. The method described herein underestimates the mean length slightly due to the probable presence of a fraction of long, and therefore undetected fragments, also in samples with mean length below 8 kb. The TRF assay overestimates the mean length, due to the fact that there is an inherited insensitivity in the Southern technique in picking up very short fragments.

One problem common to the present method and the TRF assay is the unknown length of the subtelomeric region included in the digested products. The length of the subtelomeric region even changes as a function of telomeric length, probably due to yet unknown telomere-near nucleotide modifications (Steinert, 2004). We have in the present method tried to overcome problems with the subtelomeric region by using a mixture of two frequently cutting digestion enzymes. Our data suggests that when using the chosen enzyme mix, we are able to cut relatively close to the telomere repeat block, but we most likely still have a smaller part of the subtelomeric region in our telomere-containing fragments.

In addition to the method validation presented above we have applied the method to a number of cell samples. The purpose here was not to do en extensive study, but to demonstrate applications of the method and at the same time verify previous observations, using this method. We firstly wanted to investigate if the finding by Baird et al using XpYp STELA of a few ultra short telomeres in cell samples with long mean telomere length, could be reproduced when using this method capable of detecting telomeres on all chromosome ends. We therefore investigated a number of cell samples, all telomerase-positive, but with distinctly different mean telomere length, measured by TRF-assay. The result of this series is depicted in FIG. 7. The cells are ordered after mean telomere length with cell samples with the longest length to the right. It is clear in FIG. 7, that also when using the method according to the invention we find even in a sample of cells with very long mean telomere length a subpopulation of short telomeres. These very short telomeres were not recognized before the development of the STELA technique, but they may have substantial biological relevance. They may thus very well be the reasons why senescent cells can be found in cultures of cells with very long mean telomere length, and why mean telomere lengths cannot always predict remaining population doublings of a cell culture.

In another series we compared a normal fibroblast cell line (WI38) with its ALT-positive counterpart (WI38 VA13 subline 2RA). It has long been accepted that cell lines where telomeres are maintained by the ALT-pathway have very long telomeres (20-23 kb), measured by TRF-assay and also telomeres of very diverse length. The assumption that ALT-cells have ultra-long telomeres has, however, recently been questioned by Higaki and colleagues (Higaki, 2004). They found that ALT cells actually have shorter telomeres than estimated by TRF assay, and they explained the long TRF-estimates as an artifact due to short telomeres and short ECTR forming large complexes. In order to throw light on these discrepancies we applied the method according to the invention to a fibroblast cell line and its ALT-positive counterpart. As depicted in FIG. 7 we find, in agreement with most other investigators, that the telomere length is longer in ALT-cells and that the diversity in telomere length is much higher in the ALT positive subclone than in its parental counterpart. This finding is also in agreement with earlier findings using FISH based methods to estimate individual telomere length in ALT cells.

The method according to the invention is superior to other available methods when it comes to measuring the shortest telomeres. The method only requires minute amounts of material making it possible to investigate small subpopulations of cells.

TABLE 1
Oligonucleotide sequences

| | | SEQ ID |
|---|---|---|
| Name | Sequence | NO |
| 11 +2-mer | 5′-TAC CCG CGT CCG C-3′ | 1 |
| (part of upstream oligo- | | |
| nucleotide tag) | | |
| 42-mer | 5′-TGT AGC GTG AAG ACG ACA GAA AGG GCG | 2 |
| (part of upstream oligo- | TGG TGC GGA CGC GGG-3′ | |
| nucleotide tag) | | |

Annealed upstream oligo-nucleotide tag of SEQ ID NO 1 and 2
5′-TGT AGC GTG AAG ACG ACA GAA AGG GCG TGG TGC GGA CGC GGG-3′

| 3′-CG CCT GCG CCCAT-5′ | | |
|---|---|---|
| TRCS 1 | 5′-C CCT AAC-3′ | 3 |
| (Telomere Repeat | | |
| Complementary | | |
| Sequence 1) | | |
| TRCS 2 | 5′-T AAC CCT-3 | 4 |
| TRCS 3 | 5′-C CTA ACC-3′ | 5 |
| TRCS 4 | 5′-C TAA CCC-3′ | 6 |
| TRCS 5 | 5′-A ACC CTA-3′ | 7 |
| TRCS 6 | 5′-A CCC TAA-3′ | 8 |
| down stream oligo tag 1 | 5′-TGC TCC GTG CAT CTG GCA TCC CCT AAC-3′ | 9 |
| (Telorette 1) | | |
| down stream oligo tag 2 | 5′-TGC TCC GTG CAT CTG GCA TCT AAC CCT-3′ | 10 |
| (Telorette 2) | | |
| down stream oligo tag 3 | 5′-TGC TCC GTG CAT CTG GCA TCC CTA ACC-3′ | 11 |
| (Telorette 3) | | |
| down stream oligo tag 4 | 5′-TGC TCC GTG CAT CTG GCA TCC TAA CCC-3′ | 12 |
| (Telorette 4) | | |
| down stream oligo tag 5 | 5′-TGC TCC GTG CAT CTG GCA TCA ACC CTA-3′ | 13 |
| (Telorette 5) | | |
| down stream oligo tag 6 | 5′-TGC TCC GTG CAT CTG GCA TCA CCC TAA-3′ | 14 |
| (Telorette 6) | | |
| down stream primer | 5′-TGC TCC GTG CAT CTG GCA TC-3′ | 15 |
| (Teltail) | | |
| up stream primer | 5′-TGT AGC GTG AAG ACG ACA GAA-3′ | 16 |
| (Adaptor) | | |

### REFERENCES

Allsopp, R. C., H. Vaziri, et al. (1992). “Telomere length predicts replicative capacity of human fibroblasts.” Proc Natl Acad Sci USA 89(21): 10114-8.
Baird, D. M., B. Britt-Compton, et al. (2006). “Telomere instability in the male germline.” Hum Mol Genet 15(1): 45-51.
Baird, D. M., J. Rowson, et al. (2003). “Extensive allelic variation and ultrashort telomeres in senescent human cells.” Nat Genet 33(2): 203-7.
Ben-Porath, I. and R. A. Weinberg (2004). “When cells get stressed: an integrative view of cellular senescence.” J Clin Invest 113(1): 8-13.
Benetos, A., J. P. Gardner, et al. (2004). “Short telomeres are associated with increased carotid atherosclerosis in hypertensive subjects.” Hypertension 43(2): 182-5.
Berger, M. et al., (2000), “Universal bases for hybridization, replication and chain termination”. Nucl. Acids Res. 2000 28: 2911-2914
Bischoff, C., H. Petersen, et al. (2004). “Telomere length as a predictor of survival among the elderly and oldest-old”. Ph.D. thesis
Blackburn, E. H. (2000). “Telomere states and cell fates.” Nature 408(6808): 53-6.
Broude, N. E., L. Zhang, et al. (2001). “Multiplex allele-specific target amplification based on PCR suppression.” PNAS 98(1): 206-211.
Campisi, J. (1997). “The biology of replicative senescence.” Eur J Cancer 33(5): 703-9.
Cawthon, R. M. (2002). “Telomere measurement by quantitative PCR.” Nucleic Acids Res 30(10): e47.
Cawthon, R. M., K. R. Smith, et al. (2003). “Association between telomere length in blood and mortality in people aged 60 years or older.” Lancet 361(9355): 393-5.
Collins, K. and J. R. Mitchell (2002). “Telomerase in the human organism.” Oncogene 21(4): 564-79.
Engelhardt, M., P. Drullinsky, et al. (1997). “Telomerase and telomere length in the development and progression of premalignant lesions to colorectal cancer.” Clin Cancer Res 3(11): 1931-41.
Gardner, J. P., S. Li, et al. (2005). “Rise in Insulin Resistance Is Associated With Escalated Telomere Attrition.” Circulation 111(17): 2171-2177.
Gisselsson, D., T. Jonson, et al. (2001). “Telomere dysfunction triggers extensive DNA fragmentation and evolution of complex chromosome abnormalities in human malignant tumors.” Proc Natl Acad Sci USA 98(22): 12683-8.
Griffith, J. D., L. Comeau, et al. (1999). “Mammalian telomeres end in a large duplex loop.” Cell 97(4): 503-14.
Harley, C. B., A. B. Futcher, et al. (1990). “Telomeres shorten during ageing of human fibroblasts.” Nature 345(6274): 458-60.
Higaki, T., T. Watanabe, et al. (2004). “Terminal telomere repeats are actually short in telomerase-negative immortal human cells.” Biol Pharm Bull 27(12): 1932-8.
Koppelstaetter, C., P. Jennings, et al. (2005). “Effect of tissue fixatives on telomere length determination by quantitative PCR.” Mech Ageing Dev 126(12): 1331-3.
Lavrentieva, I., N. E. Broude, et al. (1999). “High polymorphism level of genomic sequences flanking insertion sites of human endogenous retroviral long terminal repeats.” FEBS Lett 443(3): 341-7.
Martin-Ruiz, C., G. Saretzki, et al. (2004). “Stochastic Variation in Telomere Shortening Rate Causes Heterogeneity of Human Fibroblast Replicative Life Span.” J. Biol. Chem. 279(17): 17826-17833.
Meeker, A. K. and A. M. De Marzo (2004). “Recent advances in telomere biology: implications for human cancer.” Curr Opin Oncol 16(1): 32-8.
Olovnikov, A. M. (1973). “A theory of marginotomy. The incomplete copying of template margin in enzymic synthesis of polynucleotides and biological significance of the phenomenon.” J Theor Biol 41(1): 181-90.
Sfeir, A. J., W. Chai, et al. (2005). “Telomere-end processing the terminal nucleotides of human chromosomes.” Mol Cell 18(1): 131-8.
Steinert, S., J. W. Shay, et al. (2004). “Modification of subtelomeric DNA.” Mol Cell Biol 24(10): 4571-80.
Walsh, P. S., H. A. Erlich, et al. (1992). “Preferential PCR amplification of alleles: mechanisms and solutions.” PCR Methods Appl. 1(4): 241-250.
Zou, Y., A. Sfeir, et al. (2004). “Does a sentinel or a subset of short telomeres determine replicative senescence?” Mol Biol Cell 15(8): 3709-18.

1-83. (canceled)

84. A method for estimating telomere length comprising the following steps:
a) digesting a genomic DNA preparation to generate telomere fragments with an overhang using one or more restriction enzymes;
b) ligating a double-stranded upstream oligonucleotide tag comprising a double-stranded region with an overhang complementary to the overhang of the telomere fragments created by the digest of step a) and a single stranded region comprising a non-complementary sequence which is a sequence not present in the telomeric or subtelomeric region;
c) ligating a single-stranded downstream oligonucleotide tag comprising both a sequence complementary to a telomere sequence and a non-complementary sequence which is a sequence not present in the telomeric or subtelomeric region;
d) PCR amplifying telomere fragments using an upstream primer comprising a sequence identical or complementary to at least part of the non-complementary sequence or unique sequence of the upstream oligonucleotide tag and a downstream primer comprising a sequence identical or complementary to at least part of the non-complementary sequence or unique sequence of the downstream oligonucleotide tag; and
e) estimating telomere length by determining the length of the amplified telomere fragments.

85. The method according to claim 84, wherein the digestion is performed with one or more restriction enzymes that cut close to or within the subtelomeric region.

86. The method according to claim 84, wherein the one or more restriction enzymes are frequent cutters cleaving the genomic DNA into fragments of an average length of 100-500 bp.

87. The method according to claim 84, wherein
a) the double-stranded region of the upstream oligonucleotide tag is at least 5 base pairs long and at most 20 base pairs long;
b) the single stranded region of the upstream oligonucleotide tag is 15-20 nucleotides long;
c) the telomere complementary sequence of the downstream oligonucleotide tag is 4-15 nucleotides long;
d) the non-complementary sequence of the downstream oligonucleotide tag is 15-40 nucleotides long; or
e) two or more of a)-d) are present.

88. The method according to claim 87, wherein the double-stranded region of the upstream oligonucleotide tag has a CG content of at least 50%.

89. The method according to claim 84, wherein the double-stranded region of the upstream oligonucleotide tag has a Tm above 20° C. and below 60° C.

90. The method according to claim 84, wherein each strand of the upstream oligonucleotide tag is covalently bound to one strand of the telomere fragments.

91. The method according to claim 84, wherein a) the non-complementary sequence of the upstream oligonucleotide tag is a unique sequence, b) the non-complementary sequence of the downstream oligonucleotide tag is a unique sequence, or both a) and b).

92. The method according to claim 84, wherein the upstream oligonucleotide tag suppresses amplification of chromosomal DNA fragments having the upstream oligonucleotide tag in both ends.

93. The method according to claim 92, wherein the upstream oligonucleotide tag is a pair of panhandle oligonucleotides.

94. The method according to claim 84, wherein the non-complementary sequence of the downstream oligonucleotide tag is located 5′ to the telomere complementary sequence.

95. The method according to claim 84, wherein the downstream oligonucleotide tag is 18-70 nucleotides long.

96. The method according to claim 84, wherein the downstream oligonucleotide tag further comprises a sequence selected from the group consisting of sequences identified by SEQ ID NOs:3-8 or is selected from the group consisting of the sequences identified by SEQ ID NOs:9-14.

97. The method according to claim 84, wherein step b) is performed at suitable annealing conditions by lowering the temperature from 65° C. to 16° C. over an hour.

98. The method according to claim 84, wherein step c) is performed at 10-50° C. for 2-24 hours.

99. The method according to claim 84, wherein steps b) and c) are followed by an inactivation-step by heating to 65° C. for 20 minutes.

100. The method according to claim 84, wherein 5 pg-1 ng of ligated and digested genomic DNA is used as starting material in step d).

101. The method according to claim 84, wherein steps b)-d) are performed without intermediate precipitation or purification steps.

102. The method according to claim 84, wherein steps b)-d) are performed in a one-tube system.

103. The method according to claim 84, wherein the amplified telomere fragments are a) labeled by incorporation of labeled primers or labeled oligonucleotides, b) labeled using a fluorescence label, or labeled by both a) and b), and wherein length of the amplified telomere fragments is determined by use of the labels.

104. A kit for estimating telomere length according to the method of claim 84, wherein the kit comprises two or more of:
a) i) a double-stranded upstream oligonucleotide tag comprising a double-stranded region with an overhang complementary to the overhang of the telomere fragments created by the digest of step a) and a single stranded region comprising a non-complementary sequence which is a sequence not present in the telomeric or subtelomeric region and ii) a single-stranded downstream oligonucleotide tag comprising both a sequence complementary to a telomere sequence and a non-complementary sequence which is a sequence not present in the telomeric or subtelomeric region;
b) an upstream primer comprising a sequence identical or complementary to at least part of the non-complementary sequence or unique sequence of the upstream oligonucleotide tag and a downstream primer comprising a sequence identical or complementary to at least part of the non-complementary sequence or unique sequence of the downstream oligonucleotide tag;
c) one or more restriction enzymes; and
d) optionally a ligase;
e) optionally components for enzymatic reactions including PCR (NTPs, polymerase, buffers); and
f) optionally a hybridization probe.

105. The method according to claim 94, wherein a) the downstream oligonucleotide tag is identified by SEQ ID NO:1 annealed to SEQ ID NO:2, b) the downstream primer is identified by SEQ ID NO:16, or both a) and b).

106. The method according to claim 84, wherein said method is used for estimating telomere length in a biological sample or for assessing the effect of modulation of telomerase activity.

107. A method of treatment according to claim 84, wherein said method is used for
a) assessing i) a potential anti-cancer treatment, ii) another cancer related procedure, or both i) and ii);
b) assessing tolerance to a cytotoxic treatment;
c) medical diagnostics, prognostics and/or therapeutics;
d) assessing remaining proliferative capacity or lifespan of a cell;
e) assessing, treating or diagnosing male infertility; or
f) assessing the stability of stem cells in bone marrow transplantation.
